(19) United States
(12) Patent Application Publication
(10) Pub. No.: US 2015/0005184 A1
(43) Pub. Date: Jan. 1, 2015

(54) DIAGNOSIS OF MELANOMA BY NUCLEIC ACID ANALYSIS

(71) Applicant: DermTech International, La Jolla, CA (US)

(72) Inventors: John P. ALSOBROOK, Corrales, NM (US); Tara J. PALMER, San Diego, CA (US)

(73) Assignee: DermTech International, La Jolla, CA (US)

(21) Appl. No.: 14/208,155

(22) Filed: Mar. 13, 2014

Related U.S. Application Data
(60) Provisional application No. 61/841,180, filed on Jun. 28, 2013.
(60) Provisional application No. 61/841,184, filed on Jun. 28, 2013.

(51) Int. Cl. C12Q 1/68; G06F 19/00

(52) U.S. Cl. CPC C12Q 1/6886; G06F 19/34; C12Q 2600/158
USPC 506/9; 702/19

(57) ABSTRACT

The present invention provides methods for diagnosing melanoma and/or solar lentigo in a subject by analyzing nucleic acid molecules obtained from the subject. The present invention also provides methods for distinguishing melanoma from solar lentigo and/or dysplastic nevi and/or normal pigmented skin. The methods include analyzing expression or mutations in epidermal samples, of one or more skin markers. The methods can include the use of a microarray to analyze gene or protein profiles from a sample.

### CROSS-REFERENCE

This application claims the benefit of U.S. Provisional Application No. 61/841,180 and U.S. Provisional Application No. 61/841,184 both filed Jun. 28, 2013, and entitled, “DIAGNOSIS OF MELANOMA BY NUCLEIC ACID ANALYSIS,” each of which are incorporated herein by reference in their entirety.

### BACKGROUND OF THE INVENTION

Melanoma is a serious form of skin cancer in humans. It arises from the pigment cells (melanocytes), usually found in the skin. The incidence of melanoma is increasing at the fastest rate of all cancers in the United States with a lifetime risk of 1 in 68. Although melanoma accounts for only 4% of all dermatologic cancers, it is responsible for 80% of all deaths from skin cancers. The diagnosis of melanoma, when it is early stage disease, is key to its cure.

The ability to cure melanoma in its earliest skin stage, melanoma in situ (MIS), is virtually 100% if the melanoma is adequately surgically excised. If the melanoma is caught in a later stage, where it has invaded to a depth of 4 mm or more, the ten-year survival rate is less than 50%. If the melanoma is not detected until it has spread to distant parts of the body (Stage 1V), the prognosis is dismal, with only 7-9% of patients surviving 5 years, with the median survival time being 8-9 months. Tragically, the long-term “cure” rate for Stage 1V melanoma is only 1-2%.

### SUMMARY OF THE INVENTION

Described herein, in certain embodiments, are improvements for the early detection and treatment of melanoma. Diagnosis of melanoma has historically remained a difficult procedure. Previous studies have shown that even expert clinicians working in pigmented lesion clinics where melanoma is their specialty are only able to determine whether a suspicious pigmented lesion is melanoma or not with 60-80% sensitivity. This results in the need for invasive surgical biopsies of large numbers of pigmented lesions for every possible melanoma that is detected (many samples of which are later determined to not be melanoma at all), and also missed diagnosis some melanomas in their early stages.

Traditionally, melanoma has been diagnosed by biopsy and histopathological examination. To this end, approximately 20 to 30 biopsies must be performed to find one melanoma and even then some melanomas are missed in the earliest stage. The limitations of visual detection are apparent to dermatologists who are in need of ways to better determine whether suspicious lesions are melanoma or not without having to perform a biopsy first. Dermatoscopy (also known as dermoscopy or epiluminescence microscopy (ELM)) is the examination of skin lesions with a dermatoscope. This procedure traditionally consists of a magnifier (typically ×10), a non-polarized light source, a transparent plate and a liquid medium between the instrument and the skin, and allows inspection of skin lesions unobstructed by skin surface reflections. Lesions are magnified while reducing visual interference from refractive index differences at the skin-air interface. However, even skilled users of dermatoscopy have improved their ability to detect melanomas only by 5-10%. This still leads to an unacceptable sensitivity in melanoma detection and still results in the need to biopsy large numbers of benign lesions to detect a few melanomas and missed diagnoses. In addition, optical-based devices, such as MelaFind remain controversial in the field of dermatology due to concerns over false positive rates leading to unnecessary biopsies and false negatives leading to undiagnosed cancers.

Currently, there are no technologies in the U.S. or outside the U.S. that are considered the standard of care for aiding the assessment of pigmented skin lesions to the Applicants' knowledge. The methods provided herein for non-invasive gene expression tests for pigmented lesions to aid the clinician's biopsy decision are believed to be the first of its kind and have the potential to become the standard of care in melanoma diagnosis. The mechanism of analysis is uniquely different from other technologies under development and/or approved for sale in the U.S. (e.g., MelaFind, SciBase, dermoscopy). The methods provided herein are based on objective analysis of cell biological function rather than subjective pattern recognition of physical attributes of suspected skin lesions. As a result, the performance of the technology (e.g., accuracy, sensitivity and specificity) exceeds that of other technologies.

In addition, the methods described herein provide a platform that supports the development of additional diagnostic products for the clinic. For example, because the methods assess changes in cellular biology, they can be employed to identify gene expression profiles that are associated with a particular cancer stage and/or its probability of metastasis. These tests can improve the overall management of melanoma and reduce additional surgical procedures. The platform can further be adapted to other diagnostic areas in dermatology.

The methods described herein are also well-suited for clinical practice. Unlike existing device technologies, the methods provided do not require the installation and maintenance of capital equipment. Similarly, the nursing support, documentation, specimen processing, and requisition post procedure, are substantially similar to current practice. These issues are critical in a busy clinical practice where clinicians see patients every 5-7 minutes. In addition, because the methods provided involve tissue sampling, it fits within the existing physician biopsy reimbursement coding allowing physicians to receive payment for their services. By contrast, competing device technology adoption (e.g. dermoscopy or Melafind) is impeded by the lack of physician reimbursement for use of the technology.

Described herein, in certain embodiments are pigmented skin lesion assays. In some embodiments, the pigmented skin lesion assay is employed for the diagnoses of melanoma. In some embodiments, the methods provided herein address unmet needs in pigmented lesion diagnosis. For example, the pigmented lesion assays provided herein demonstrate a high accuracy (e.g., at least about 80%, at least about 90%, at least about 93%, at least about 95%, or greater) in differentiating melanoma from non-melanoma using histopathology as a reference standard. In certain instances, the high sensitivity of the methods provided herein will improve the clinician's ability to identify pigmented lesions that are demonstrating malignant changes so that they can be surgically biopsied for histopathologic diagnosis. In certain instances, the high specificity will minimize the number of surgical biopsies required to identify these melanomas. In some embodiments, the negative predictive value of the assays provided exceeds about 98%, about 98.5%, about 99%, or about 99.5%. Thus, in some embodiments, the probability of a false negative test is less than about 2%, less than about 1.5%, less than about 1%, or less than about 0.5%.

In some embodiments, the assays provided herein analyze the expression of at least 2 genes in tissue samples collected using an adhesive patch. In some embodiments, relative to non-melanoma skin lesions, one of the two genes is down-regulated and the one of the two genes is up-regulated. In some embodiments, analysis of the two genes allows for accurate identification of gene expression consistent with melanoma.

In some embodiments, the samples for the assays described herein are obtained via non-invasive methods. In some embodiments, the samples are obtained using an adhesive patch applied to the skin. Because the assays provided herein are, at least in certain embodiments, performed on samples collected using non-invasive methods, the pigmented skin lesion assays provided herein provide a valuable non-invasive tool for the clinician to determine the need for one or more surgical biopsies. As demonstrated herein, including but not limited to in the examples provided, the pigmented skin lesion assays described herein have high sensitivity and high specificity. Practice of the assays provided herein, in certain embodiments, substantially reduces the number of unnecessary surgical biopsies performed. In addition, because the assays provided herein demonstrate a high sensitivity, the accuracy of diagnosis in lesions that are sent to pathology is improved which provides greater patient care.

The development of the pigmented lesion assays described herein has involved analysis of over 500 lesion samples, including over 250 melanomas. The performance of the 2-gene expression profile classifier, which is one embodiment of the subject matter described herein, has been elucidated in a series of 3 clinical studies examining 288 lesions with over 140 melanomas. The PLA score, which is determined from the expression levels of these two genes, steadily increases from non-melanoma lesions, to melanoma in situ (MIS), to invasive melanoma, demonstrating the progression of the gene expression as the cancer advances. The average score of a melanoma and melanoma in situ is statistically significantly different from non-melanoma (p<<0.00001). In two validation studies using histopathology as the reference standard, the 2-gene expression profile demonstrated 93% accuracy in identifying non-melanoma and melanoma lesions, with a sensitivity of 93% and a specificity of 90%. At a projected melanoma prevalence of 7%, the negative predictive value was greater than 99%.

Because of the extremely high cure rate of melanoma in situ, it is important to accurately differentiate this stage from invasive melanoma. Additionally, the surgical treatment and follow on tests are more extensive for invasive melanoma versus melanoma in situ. Subtle changes in the way tissue is processed and the anatomic architecture of pigmented lesions can obscure accurate differentiation of these stages. Just as the cancer transformation of benign cells is controlled by gene expression, the development of invasive tumor properties is also controlled by gene expression. Provided herein are gene expression profiles for lesions that have become invasive versus those that are melanoma in situ. As demonstrated herein in a study involving 100 lesions, practice of the methods provided herein accurately differentiates those melanomas that are invasive versus those that are melanoma in situ.

The inventors have identified a need for further development of technology that will enable physicians to determine the nature and extent of suspicious lesions of the skin. Such technology is directed to assay of the physiology of the patient's lesion based on gene expression profiles to enable a sensitive and accurate diagnosis of skin lesion.

Described herein, in some embodiments, is a method for characterizing a skin lesion in a subject, comprising analyzing a nucleic acid molecule from one or more genes comprising: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, or VIM, in a sample of the skin lesion, thereby producing a characterization of the skin lesion. In some embodiments, LINC00518 is analyzed. In some embodiments, the genes are selected from LINC00518, TRIB2, KIT, SDCBP, TYR, NAMPT, ACTN4, EDNRB, GPM6B, CNN2, MCOLN3, PRAME, TMEM80, TTC3, and CMIP, and combinations thereof. In some embodiments, the genes are selected from LINC00518, CMIP, ACTN4, TMEM80 and NAMPT and combinations thereof. In some embodiments, the one or more genes are selected from LINC00518 and CMIP, and combinations thereof. In some embodiments, the combination of LINC00518 and CMIP is analyzed. In some embodiments, the one or more genes are selected from LINC00518 and TMEM80, and combinations thereof. In some embodiments, the combination of LINC00518 and TMEM80 is analyzed. In some embodiments, the one of more genes are selected from LINC00518 and ACTN4, and combinations thereof. In some embodiments, the combination of LINC00518 and ACTN4 are analyzed. In some embodiments, the nucleic acid molecule comprises RNA. In some embodiments, analyzing the nucleic acid molecule comprises detecting one or more mutations in the nucleic acid sequence of the nucleic acid molecule. In some embodiments, the one or more mutations are selected from the group consisting of a substitution, a deletion, and an insertion. In some embodiments, the method further comprises amplifying the nucleic acid molecule obtained from the sample prior to analyzing. In some embodiments, the sample is obtained by applying an adhesive tape to a target area of skin in a manner sufficient to isolate the sample adhering to the adhesive tape. In some embodiments, the tape comprises a rubber adhesive on a polyurethane film. In some embodiments, about one to ten adhesive tapes or one to ten applications of a tape are applied and removed from the skin. In some embodiments, the method further comprises using the characterization to determine a treatment regimen. In some embodiments, the nucleic acid molecule, or an amplification product thereof, is applied to a microarray. In some embodiments, analyzing a nucleic acid molecule comprises detecting an expression profile. In some embodiments, the expression profile is detected using a microarray. In some embodiments, the sample is obtained from a biopsy taken at the site of the skin lesion or surrounding margin. In some embodiments, the method further comprises taking a biopsy of the target area of the skin. In some embodiments, the analyzing is performed in situ.

Disclosed herein, in some embodiments, is a method of distinguishing melanoma from dysplastic nevi or normal pigmented skin in a subject, comprising analyzing a nucleic acid molecule from one or more genes selected from: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, and VIM, in a sample from the subject, thereby distinguishing melanoma from dysplastic nevi or normal pigmented skin in a subject. In some embodiments, the one or more genes are selected from LINC00518, TRIB2, KIT, SDCBP, TYR, NAMPT, ACTN4, EDNRB, GPM6B, CNN2, MCOLN3, PRAME, TMEM80, TTC3, and CMIP, and combinations thereof. In some embodiments, one or more genes is selected from: ACTN4, LINC00518, CMIP, TTC3, TMEM80, HLA-C, and PRAME, and combinations thereof. In some embodiments, two or more genes are selected from: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, and VIM, and combinations thereof. In some embodiments, the two or more genes are selected from LINC00518, TRIB2, KIT, SDCBP, TYR, NAMPT, ACTN4, EDNRB, GPM6B, CNN2, MCOLN3, PRAME, TMEM80, TTC3, and CMIP, and combinations thereof. In some embodiments, the two or more genes is selected from: ACTN4, LINC00518, CMIP, TTC3, TMEM80, HLA-C, and PRAME. In some embodiments, the genes analyzed are selected from the group consisting of LINC00518, CMIP, ACTN4, TMEM80 and NAMPT and combinations thereof. In some embodiments, the genes analyzed are selected from the group consisting of ACTN4, LINC00518, CMIP, TTC3, TMEM80, HLA-C, and PRAME and combinations thereof. In some embodiments, LINC00518 is analyzed. In some embodiments, the two or more genes comprise LINC00518 and CMIP, and combinations thereof. In some embodiments, the combination of LINC00518 and CMIP is analyzed. In some embodiments, the two or more genes comprise LINC00518 and TMEM80, and combinations thereof. In some embodiments, the combination of LINC00518 and TMEM80 is analyzed. In some embodiments, the two or more genes comprise LINC00518 and ACTN4, and combinations thereof. In some embodiments, the combination of LINC00518 and ACTN4 are analyzed. In some embodiments, the two or more genes comprise LINC00518 and NAMPT, and combinations thereof. In some embodiments, the combination of LINC00518 and NAMPT are analyzed. In some embodiments, the two or more genes comprise LINC00518 and TTC3, and combinations thereof. In some embodiments, the combination of LINC00518 and TTC3 are analyzed. In some embodiments, the two or more genes comprise LINC00518 and HLA-C, and combinations thereof. In some embodiments, the combination of LINC00518 and HLA-C are analyzed. In some embodiments, the two or more genes comprise LINC00518 and PRAME, and combinations thereof. In some embodiments, the combination of LINC00518 and PRAME are analyzed. In some embodiments, the two or more genes comprise ACTN4, LINC00518 and PRAME, and combinations thereof. In some embodiments, the combination of ACTN4, LINC00518 and PRAME are analyzed. In some embodiments, the two or more genes comprise LINC00518 and TRIB2, and combinations thereof. In some embodiments, the two or more genes comprise LINC00518 and TRIB2, and combinations thereof. In some embodiments, the two or more genes comprise LINC00518 and GPM6B, and combinations thereof. In some embodiments, the combination of LINC00518 and GPM6B is analyzed. In some embodiments, the two or more genes comprise TRIB2 and NAMPT, and combinations thereof. In some embodiments, the combination of TRIB2 and NAMPT is analyzed. In some embodiments, the two or more genes comprise TRIB2 and KIT, and combinations thereof. In some embodiments, the combination of TRIB2 and KIT is analyzed. In some embodiments, the combination of TRIB2 and ACTN4 is analyzed. In some embodiments, the combination of TRIB2 and ACTN4 is analyzed.

Disclosed herein, in some embodiments, is a method of diagnosing melanoma, comprising analyzing a nucleic acid molecule from one or more genes selected from: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, and VIM, in a sample from the subject, thereby diagnosing the presence or absence of melanoma in a subject. In some embodiments, the one or more genes are selected from LINC00518, TRIB2, KIT, SDCBP, TYR, NAMPT, ACTN4, EDNRB, GPM6B, CNN2, MCOLN3, PRAME, TMEM80, TTC3, and CMIP, and combinations thereof. In some embodiments, one or more genes is selected from: ACTN4, LINC00518, CMIP, TTC3, TMEM80, HLA-C, and PRAME, and combinations thereof. In some embodiments, two or more genes are selected from: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, and VIM, and combinations thereof. In some embodiments, the two or more genes are selected from LINC00518, TRIB2, KIT, SDCBP, TYR, NAMPT, ACTN4, EDNRB, GPM6B, CNN2, MCOLN3, PRAME, TMEM80, TTC3, and CMIP, and combinations thereof. In some embodiments, the two or more genes is selected from: ACTN4, LINC00518, CMIP, TTC3, TMEM80, HLA-C, and PRAME. In some embodiments, the genes analyzed are selected from the group consisting of LINC00518, CMIP, ACTN4, TMEM80 and NAMPT and combinations thereof. In some embodiments, the genes analyzed are selected from the group consisting of ACTN4, LINC00518, CMIP, TTC3, TMEM80, HLA-C, and PRAME and combinations thereof. In some embodiments, LINC00518 is analyzed. In some embodiments, the two or more genes comprise LINC00518 and CMIP, and combinations thereof. In some embodiments, the combination of LINC00518 and CMIP is analyzed. In some embodiments, the two or more genes comprise LINC00518 and TMEM80, and combinations thereof. In some embodiments, the combination of LINC00518 and TMEM80 is analyzed. In some embodiments, the two or more genes comprise LINC00518 and ACTN4, and combinations thereof. In some embodiments, the combination of LINC00518 and ACTN4 are analyzed. In some embodiments, the two or more genes comprise LINC00518 and NAMPT, and combinations thereof. In some embodiments, the combination of LINC00518 and NAMPT are analyzed. In some embodiments, the two or more genes comprise LINC00518 and TTC3, and combinations thereof. In some embodiments, the combination of LINC00518 and TTC3 are analyzed. In some embodiments, the two or more genes comprise LINC00518 and HLA-C, and combinations thereof. In some embodiments, the combination of LINC00518 and HLA-C are analyzed. In some embodiments, the two or more genes comprise LINC00518 and PRAME, and combinations thereof. In some embodiments, the combination of LINC00518 and PRAME are analyzed. In some embodiments, the two or more genes comprise ACTN4, LINC00518 and PRAME, and combinations thereof. In some embodiments, the combination of ACTN4, LINC00518 and PRAME are analyzed. In some embodiments, the two or more genes comprise LINC00518 and TRIB2, and combinations thereof. In some embodiments, the two or more genes comprise LINC00518 and TRIB2, and combinations thereof. In some embodiments, the two or more genes comprise LINC00518 and GPM6B, and combinations thereof. In some embodiments, the combination of LINC00518 and GPM6B is analyzed. In some embodiments, the two or more genes comprise TRIB2 and NAMPT, and combinations thereof. In some embodiments, the combination of TRIB2 and NAMPT is analyzed. In some embodiments, the two or more genes comprise TRIB2 and KIT, and combinations thereof. In some embodiments, the combination of TRIB2 and KIT is analyzed. In some embodiments, the combination of TRIB2 and ACTN4 is analyzed. In some embodiments, the combination of TRIB2 and ACTN4 is analyzed.

Disclosed herein, in some embodiments, is a method for diagnosing solar lentigo, comprising analyzing a nucleic acid molecule from one or more genes selected from: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, and VIM, in a sample from the subject, thereby diagnosing the presence or absence of solar lentigo in a subject. In some embodiments, the one or more genes are selected from LINC00518, TRIB2, KIT, SDCBP, TYR, NAMPT, ACTN4, EDNRB, GPM6B, CNN2, MCOLN3, PRAME, TMEM80, TTC3, and CMIP, and combinations thereof. In some embodiments, one or more genes is selected from: ACTN4, LINC00518, CMIP, TTC3, TMEM80, HLA-C, and PRAME, and combinations thereof. In some embodiments, two or more genes are selected from: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, and VIM, and combinations thereof. In some embodiments, the two or more genes are selected from LINC00518, TRIB2, KIT, SDCBP, TYR, NAMPT, ACTN4, EDNRB, GPM6B, CNN2, MCOLN3, PRAME, TMEM80, TTC3, and CMIP, and combinations thereof. In some embodiments, the two or more genes is selected from: ACTN4, LINC00518, CMIP, TTC3, TMEM80, HLA-C, and PRAME. In some embodiments, the genes analyzed are selected from the group consisting of LINC00518, CMIP, ACTN4, TMEM80 and NAMPT and combinations thereof. In some embodiments, the genes analyzed are selected from the group consisting of ACTN4, LINC00518, CMIP, TTC3, TMEM80, HLA-C, and PRAME and combinations thereof. In some embodiments, LINC00518 is analyzed. In some embodiments, the two or more genes comprise LINC00518 and CMIP, and combinations thereof. In some embodiments, the combination of LINC00518 and CMIP is analyzed. In some embodiments, the two or more genes comprise LINC00518 and TMEM80, and combinations thereof. In some embodiments, the combination of LINC00518 and TMEM80 is analyzed. In some embodiments, the two or more genes comprise LINC00518 and ACTN4, and combinations thereof. In some embodiments, the combination of LINC00518 and ACTN4 are analyzed. In some embodiments, the two or more genes comprise LINC00518 and NAMPT, and combinations thereof. In some embodiments, the combination of LINC00518 and NAMPT are analyzed. In some embodiments, the two or more genes comprise LINC00518 and TTC3, and combinations thereof. In some embodiments, the combination of LINC00518 and TTC3 are analyzed. In some embodiments, the two or more genes comprise LINC00518 and HLA-C, and combinations thereof. In some embodiments, the combination of LINC00518 and HLA-C are analyzed. In some embodiments, the two or more genes comprise LINC00518 and PRAME, and combinations thereof. In some embodiments, the combination of LINC00518 and PRAME are analyzed. In some embodiments, the two or more genes comprise ACTN4, LINC00518 and PRAME, and combinations thereof. In some embodiments, the combination of ACTN4, LINC00518 and PRAME are analyzed. In some embodiments, the two or more genes comprise LINC00518 and TRIB2, and combinations thereof. In some embodiments, the two or more genes comprise LINC00518 and TRIB2, and combinations thereof. In some embodiments, the two or more genes comprise LINC00518 and GPM6B, and combinations thereof. In some embodiments, the combination of LINC00518 and GPM6B is analyzed. In some embodiments, the two or more genes comprise TRIB2 and NAMPT, and combinations thereof. In some embodiments, the combination of TRIB2 and NAMPT is analyzed. In some embodiments, the two or more genes comprise TRIB2 and KIT, and combinations thereof. In some embodiments, the combination of TRIB2 and KIT is analyzed. In some embodiments, the combination of TRIB2 and ACTN4 is analyzed. In some embodiments, the combination of TRIB2 and ACTN4 is analyzed.

Disclosed herein, in some embodiments, is a system for characterizing a skin lesion in a subject, comprising analyzing a nucleic acid molecule from one or more genes comprising: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, or VIM, in a sample of the skin lesion, thereby producing a characterization of the skin lesion. In some embodiments, the one or more genes are selected from LINC00518, TRIB2, KIT, SDCBP, TYR, NAMPT, ACTN4, EDNRB, GPM6B, CNN2, MCOLN3, PRAME, TMEM80, TTC3, and CMIP, and combinations thereof. In some embodiments, one or more genes is selected from: ACTN4, LINC00518, CMIP, TTC3, TMEM80, HLA-C, and PRAME, and combinations thereof. In some embodiments, two or more genes are selected from: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, and VIM, and combinations thereof. In some embodiments, the two or more genes are selected from LINC00518, TRIB2, KIT, SDCBP, TYR, NAMPT, ACTN4, EDNRB, GPM6B, CNN2, MCOLN3, PRAME, TMEM80, TTC3, and CMIP, and combinations thereof. In some embodiments, the two or more genes is selected from: ACTN4, LINC00518, CMIP, TTC3, TMEM80, HLA-C, and PRAME. In some embodiments, the genes analyzed are selected from the group consisting of LINC00518, CMIP, ACTN4, TMEM80 and NAMPT and combinations thereof. In some embodiments, the genes analyzed are selected from the group consisting of ACTN4, LINC00518, CMIP, TTC3, TMEM80, HLA-C, and PRAME and combinations thereof. In some embodiments, LINC00518 is analyzed. In some embodiments, the two or more genes comprise LINC00518 and CMIP, and combinations thereof. In some embodiments, the combination of LINC00518 and CMIP is analyzed. In some embodiments, the two or more genes comprise LINC00518 and TMEM80, and combinations thereof. In some embodiments, the combination of LINC00518 and TMEM80 is analyzed. In some embodiments, the two or more genes comprise LINC00518 and ACTN4, and combinations thereof. In some embodiments, the combination of LINC00518 and ACTN4 are analyzed. In some embodiments, the two or more genes comprise LINC00518 and NAMPT, and combinations thereof. In some embodiments, the combination of LINC00518 and NAMPT are analyzed. In some embodiments, the two or more genes comprise LINC00518 and TTC3, and combinations thereof. In some embodiments, the combination of LINC00518 and TTC3 are analyzed. In some embodiments, the two or more genes comprise LINC00518 and HLA-C, and combinations thereof. In some embodiments, the combination of LINC00518 and HLA-C are analyzed. In some embodiments, the two or more genes comprise LINC00518 and PRAME, and combinations thereof. In some embodiments, the combination of LINC00518 and PRAME are analyzed. In some embodiments, the two or more genes comprise ACTN4, LINC00518 and PRAME, and combinations thereof. In some embodiments, the combination of ACTN4, LINC00518 and PRAME are analyzed. In some embodiments, the two or more genes comprise LINC00518 and TRIB2, and combinations thereof. In some embodiments, the two or more genes comprise LINC00518 and TRIB2, and combinations thereof. In some embodiments, the two or more genes comprise LINC00518 and GPM6B, and combinations thereof. In some embodiments, the combination of LINC00518 and GPM6B is analyzed. In some embodiments, the two or more genes comprise TRIB2 and NAMPT, and combinations thereof. In some embodiments, the combination of TRIB2 and NAMPT is analyzed. In some embodiments, the two or more genes comprise TRIB2 and KIT, and combinations thereof. In some embodiments, the combination of TRIB2 and KIT is analyzed. In some embodiments, the combination of TRIB2 and ACTN4 is analyzed. In some embodiments, the combination of TRIB2 and ACTN4 is analyzed.

Disclosed herein, in some embodiments, is a system for characterizing a skin lesion in a subject in a subject, comprising: (a) a computer processing device, optionally connected to a computer network; and (b) a software module executed by the computer processing device to analyzing a nucleic acid molecule from one or more genes selected from: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, in a sample from a skin lesion, to a standard or control. In some embodiments, the one or more genes are selected from LINC00518, TRIB2, KIT, SDCBP, TYR, NAMPT, ACTN4, EDNRB, GPM6B, CNN2, MCOLN3, PRAME, TMEM80, TTC3, and CMIP, and combinations thereof. In some embodiments, one or more genes is selected from: ACTN4, LINC00518, CMIP, TTC3, TMEM80, HLA-C, and PRAME, and combinations thereof. In some embodiments, two or more genes are selected from: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, and VIM, and combinations thereof. In some embodiments, the two or more genes are selected from LINC00518, TRIB2, KIT, SDCBP, TYR, NAMPT, ACTN4, EDNRB, GPM6B, CNN2, MCOLN3, PRAME, TMEM80, TTC3, and CMIP, and combinations thereof. In some embodiments, the two or more genes is selected from: ACTN4, LINC00518, CMIP, TTC3, TMEM80, HLA-C, and PRAME. In some embodiments, the genes analyzed are selected from the group consisting of LINC00518, CMIP, ACTN4, TMEM80 and NAMPT and combinations thereof. In some embodiments, the genes analyzed are selected from the group consisting of ACTN4, LINC00518, CMIP, TTC3, TMEM80, HLA-C, and PRAME and combinations thereof. In some embodiments, LINC00518 is analyzed. In some embodiments, the two or more genes comprise LINC00518 and CMIP, and combinations thereof. In some embodiments, the combination of LINC00518 and CMIP is analyzed. In some embodiments, the two or more genes comprise LINC00518 and TMEM80, and combinations thereof. In some embodiments, the combination of LINC00518 and TMEM80 is analyzed. In some embodiments, the two or more genes comprise LINC00518 and ACTN4, and combinations thereof. In some embodiments, the combination of LINC00518 and ACTN4 are analyzed. In some embodiments, the two or more genes comprise LINC00518 and NAMPT, and combinations thereof. In some embodiments, the combination of LINC00518 and NAMPT are analyzed. In some embodiments, the two or more genes comprise LINC00518 and TTC3, and combinations thereof. In some embodiments, the combination of LINC00518 and TTC3 are analyzed. In some embodiments, the two or more genes comprise LINC00518 and HLA-C, and combinations thereof. In some embodiments, the combination of LINC00518 and HLA-C are analyzed. In some embodiments, the two or more genes comprise LINC00518 and PRAME, and combinations thereof. In some embodiments, the combination of LINC00518 and PRAME are analyzed. In some embodiments, the two or more genes comprise ACTN4, LINC00518 and PRAME, and combinations thereof. In some embodiments, the combination of ACTN4, LINC00518 and PRAME are analyzed. In some embodiments, the two or more genes comprise LINC00518 and TRIB2, and combinations thereof. In some embodiments, the two or more genes comprise LINC00518 and TRIB2, and combinations thereof. In some embodiments, the two or more genes comprise LINC00518 and GPM6B, and combinations thereof. In some embodiments, the combination of LINC00518 and GPM6B is analyzed. In some embodiments, the two or more genes comprise TRIB2 and NAMPT, and combinations thereof. In some embodiments, the combination of TRIB2 and NAMPT is analyzed. In some embodiments, the two or more genes comprise TRIB2 and KIT, and combinations thereof. In some embodiments, the combination of TRIB2 and KIT is analyzed. In some embodiments, the combination of TRIB2 and ACTN4 is analyzed. In some embodiments, the combination of TRIB2 and ACTN4 is analyzed.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 exemplifies the contribution of genes (along the x-axis) including GPM6B, TYR, KIT, TTC3, TMEM80, NAMPT, CNN2, MCOLN3, SDCBP, CMIP, ACTN4, EDNRB, LINC00518 and TRIB2 in identification of melanoma.

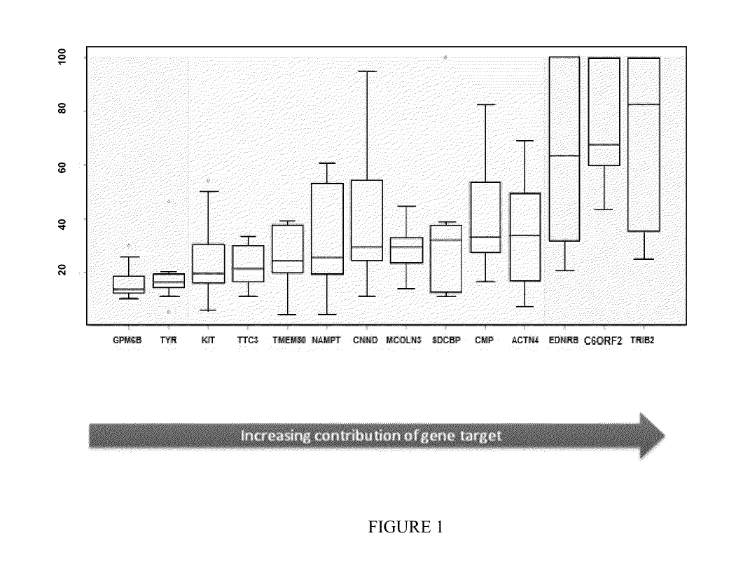

FIG. 2 exemplifies the AUC-ROC of all two gene combinations from the 15 gene set of GPM6B, TYR, KIT, TTC3, TMEM80, NAMPT, CNN2, MCOLN3, SDCBP, CMIP, ACTN4, EDNRB, LINC00518, TRIB2 and PRAME. The datapoint to the farthest right (with an AUC ROC value of nearly 0.95) represents the combination of LINC00518 and CMIP.

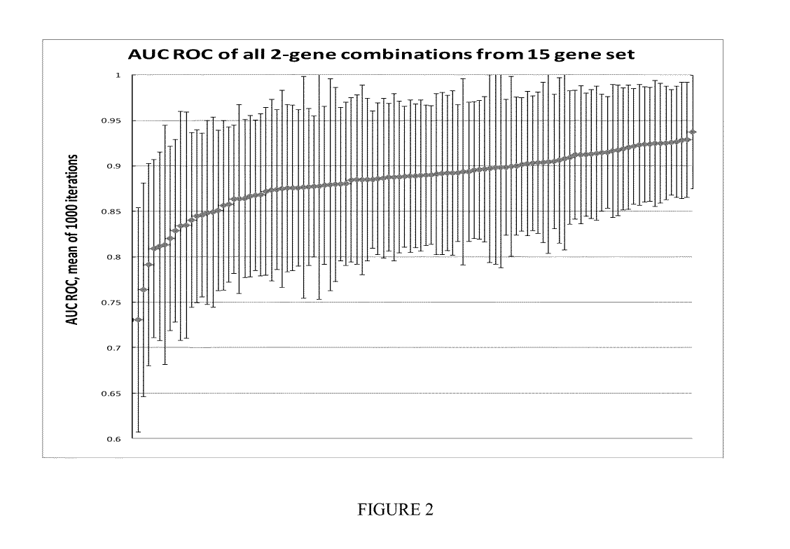

FIG. 3 exemplifies the Latin Square Experimental design, as described in Example 3.

FIG. 4 exemplifies the sensitivity and linear dynamic range of the CMIP gene detected on the OpenArray plate.

FIG. 5 exemplifies a comparative assessment of the performance of a multi-gene biomarker for melanoma on the TaqMan OA and microarray platforms, as described in Example 3.

FIG. 6 exemplifies performance of gene pairs from the 15-Gene classifier for diagnosing melanoma. FIG. 6 depicts a histogram of all 105 pairwise combinations from the 15 genes classifier melanoma signature, binned by the AUC-ROC for each pair. The width on the x-axis of each column represents the lower and upper limits of the AUC-ROC for that bin. The number of genes in each bin is represented by the height of the column on the y-axis.

FIG. 7 exemplifies the case distribution of classifier scores for Set B. FIG. 7 depicts distribution of Set B cases scored by the Set A classifier. The x-axis is the case number. The y-axis represents the algorithmic score based on expression levels of a 2-gene classifier. Nevi are plotted on the left as closed circles (). Melanomas are plotted on the right as closed triangles (▴).

FIG. 8 exemplifies the case distribution of classifier scores for Set A. FIG. 8 depicts distribution of Set A cases scored by the Set B classifier. The x-axis is the case number. The y-axis represents the algorithmic score based on expression levels of a 2-gene classifier. Nevi are plotted on the left as closed circles (). Melanomas are plotted on the right as closed triangles (▴).

FIG. 9 exemplifies gene classifier validation for LINC00518 and CMIP using 1000 pg of RNA as input. The AUC was found to be 0.96, the sensitivity was found to be 99% and the specificity was found to be 83%.

FIG. 10 exemplifies gene classifier validation for the gene pair combination of LINC00518 and CMIP in classifying melanoma, using 250 pg of RNA as input. The sensitivity was found to be 95%, while the specificity was found to be 70%.

FIG. 11 exemplifies gene classifier validation for the gene pair combination of LINC00518 and CMIP in classifying melanoma, using 100 pg of RNA as input. At a threshold of 0.25, the sensitivity was 75%, whereas at a threshold of 0.18, the sensitivity was found to be 84%.

FIG. 12 exemplifies a gene classifier validation for the gene pair combination of LINC00518 and CMIP in classifying non-melanoma, melanoma in-situ, and invasive melanoma. The mean of the algorithmic score is shown for each set of specimens in the validation study, with error bars indicating the 95% confidence interval of that mean. A one-way analysis of variance shows a significant difference of the algorithmic score among the 3 groups, with a p-value<<0.0001.

### DETAILED DESCRIPTION OF THE INVENTION

There are two main motivations for conducting genome wide expression profiling studies in melanoma. First, melanoma is one of the best characterized carcinogenesis models for gradual progression of benign lesions to cancer: normal pigmented cells to nevi to atypical nevi to primary melanoma in situ to invasive primary melanoma to aggressive metastatic melanoma. This progression is known to correlate with distinctive chromosomal changes, and is thought to be mediated by stepwise progressive changes in gene expression, suggesting that expression profiling may identify genes responsible for tumorigenesis in melanoma. Indeed, candidate tumor genes have been identified with microarray analyses of melanoma cell lines. The second reason is that molecular characterization of tumors may allow a better staging classification of tumors and prognosis prediction. While histological characteristics such as the thickness and ulceration of tumors have some value as predictors of prognosis, there is lack of informative markers that help determine which patients will do well and which patients will have progressive disease and metastasis. Molecular markers identified in microarray experiments of tumors are already being introduced into clinical practice in the management of breast cancer. Gene expression profiling experiments in melanoma and melanoma cell lines suggest that the classification of melanoma can be improved, but studies are lacking with sufficient power to define molecular criteria for diagnosis or identify prognostic markers; the establishments of such markers would represent a major advance in melanoma care. A major reason for the lack of powerful microarray studies in melanoma is that, unlike most solid tumors, it is necessary to paraffin embed and section the whole lesion for histology, leaving no sample for RNA isolation. Although this situation is now changing, the ability to avoid biopsy until a definitive diagnosis is made would be powerful for subjects that would not normally be eligible for one or more biopsies.

Also provided herein is a method for diagnosing a disease state by establishing a gene expression pattern of a target area suspected of being melanoma on the skin of a subject and comparing the subject's gene expression profile to a reference gene expression profile obtained from a corresponding normal skin sample. In one embodiment, the target area of the skin simultaneously expresses a plurality of genes at the protein level that are markers for melanoma.

In another aspect, the methods of the present invention can also be useful for monitoring the progression of diseases and the effectiveness of treatments. For example, by comparing the projected profile prior to treatment with the profile after treatment.

In a related aspect, the methods of the present invention can also be useful for determining an appropriate treatment regimen for a subject having a specific cancer or melanoma. In another related aspect, the methods of the present invention can also be useful for determining an appropriate treatment regimen for a subject having solar lentigo. Thus, the methods of the invention are useful for providing a means for practicing personalized medicine, wherein treatment is tailored to a subject based on the particular characteristics of the cancer or skin lesion in the subject. The method can be practiced, for example, by first determining whether the skin lesion is melanoma or solar lentigo, as described above.

In one embodiment, the method involves use of a non-invasive approach for recovering nucleic acids such as DNA or messenger RNA or proteins from the surface of skin via a tape stripping procedure that permits a direct quantitative and qualitative assessment of biomarkers. Although tape-harvested nucleic acid and protein expression products are shown to be comparable in quality and utility to recovering such molecules by biopsy, the non-invasive method provides information regarding cells of the outermost layers of the skin that may not be obtained using biopsy samples. Finally, the non-invasive method is far less traumatic than a biopsy.

Thus, the non-invasive method is used to capture cells on pigmented skin lesions that are suspected of being melanomas. Nucleic acid molecules obtained from skin cells captured by the non-invasive method are analyzed in order to diagnose the nature of the lesion (e.g., malignant melanoma). In one embodiment, a nucleic acid molecule is amplified prior to analysis. Secondary outcomes could include tests for diagnosis and prognosis of a variety of pigmented skin lesions and even to predict a therapeutic regimen. In another embodiment, the skin cells are lysed to extract one or more proteins, which are then quantitated to diagnose the nature of the lesion. It should be understood that the methods of the invention are not limited to non-invasive techniques for obtaining skin samples. For example, but not by limitation, one of skill in the art would know other techniques for obtaining a skin sample such as scraping of the skin, biopsy, suction, blowing and other techniques. As described herein, non-invasive tape stripping is an illustrative example for obtaining a skin sample.

### CERTAIN TERMINOLOGY

As used herein, the term “skin lesion” refers to a change in the color or texture in an area of skin. As such, “skin lesions suspected of being melanoma” are skin lesions with characteristics of malignant melanoma, which are well known to those of skill in the art, such as dermatologists and oncologists. Such lesions are sometimes raised and can have a color that is different from the color of normal skin of an individual (e.g., brown, black, red, or blue). Lesions suspected of being melanoma sometimes include a mixture of colors, are often asymmetrical, can change in appearance over time, and may bleed. A skin lesion suspected of being melanoma may be a mole or nevus. Melanoma lesions are usually, but not always, larger than 6 mm in diameter. Melanoma includes superficial spreading melanoma, nodular melanoma, acral lentiginous melanoma, and lentigo maligna melanoma. The term “lentigo maligna” refers to a precancerous lesion on the skin, especially in areas exposed to the sun, that is flat, mottled, and brownish with an irregular outline and grows slowly over a period of years. Melanoma can occur on skin that has been overexposed to the sun. Therefore, in one embodiment the skin sample is taken from an area of skin that has been overexposed to the sun.

The term “melanoma” includes, but is not limited to, melanomas, metastatic melanomas, melanomas derived from either melanocytes or melanocyte related nevus cells, melanocarcinomas, melanoepitheliomas, melanosarcomas, melanoma in situ, superficial spreading melanoma, nodular melanoma, lentigo maligna melanoma, acral lentiginous melanoma, invasive melanoma or familial atypical mole and melanoma (FAMM) syndrome.

The term “dysplastic nevus” refers to an atypical mole or a mole whose appearance is different from that of common moles. Dysplastic nevi are generally larger than ordinary moles and have irregular and indistinct borders. Their color frequently is not uniform and ranges from pink to dark brown; they usually are flat, but parts may be raised above the skin surface. Dysplastic naevus can be found anywhere, but are most common on the trunk of a subject.

As used herein, the term “gene” refers to a linear sequence of nucleotides along a segment of DNA that provides the coded instructions for synthesis of RNA, which, when translated into protein, leads to the expression of hereditary character. As such, the term “skin marker” or “biomarker” refers to a gene whose expression level is different between skin surface samples at the site of malignant melanoma and skin surface samples of normal skin or a lesion, which is benign, such as a benign nevus. Therefore, expression of a melanoma skin marker of the invention is related to, or indicative of, melanoma. Many statistical techniques are known in the art, which can be used to determine whether a statistically significant difference in expression is observed at a high (e.g., 90% or 95%) confidence level. As such, an increase or decrease in expression of these genes is related to and can characterize malignant melanoma. In one embodiment, there is at least a two-fold difference in levels between skin sample near the site of malignant melanoma and skin samples from normal skin.

As used herein, “gene product” means any product expressed by a gene, including nucleic acids or polypeptides. In some embodiments, a gene product is a transcribed nucleic acid, such as RNA. In some embodiments, the RNA is a coding RNA, e.g. a messenger RNA (mRNA). In some embodiments, the RNA is a non-coding RNA. In some embodiments, the non-coding RNA is a transfer RNA (tRNA), ribosomal RNA (rRNA), snoRNA, microRNA, siRNA, snRNA, exRNA, piRNA and long ncRNA. In some embodiments, a gene product is a protein that is translated from and expressed mRNA or other nucleic acid.

As used herein, the term “sample” refers to any preparation derived from skin of a subject. In some embodiments, a sample of cells obtained using the non-invasive method described herein is used to isolate polynucleotides, polypeptides, and/or lipids, for the methods provided herein. In some embodiments, samples for the methods provided herein are taken from a skin lesion that is suspected of being the result of a disease or a pathological state, such as melanoma. In some embodiments, samples are taken of the skin surface of the suspicious lesion using non-invasive skin sampling methods described herein.

As used herein, the term “skin” refers to the outer protective covering of the body, consisting of the corium and the epidermis, and is understood to include sweat and sebaceous glands, as well as hair follicle structures. As used herein, the term “cutaneous” refers generally to attributes of the skin, as appropriate to the context in which they are used. In some embodiments, the skin is mammalian skin. In some embodiments, the skin is human skin.

As used herein, the term “ameliorating” or “treating” means that the clinical signs and/or the symptoms associated with the cancer or melanoma are lessened as a result of the actions performed. The signs or symptoms to be monitored will be characteristic of a particular cancer or melanoma and will be well known to the skilled clinician, as will the methods for monitoring the signs and conditions. Thus, a “treatment regimen” refers to any systematic plan or course for treating a disease or cancer in a subject.

### Methods for Obtaining a Skin Sample

Samples from a tissue can be isolated by any number of means well known in the art. Invasive methods for isolating a sample include, but are not limited to the use of needles or scalpels, for example during biopsies of various tissues. Non-invasive methods for isolating a sample include, but are not limited to tape-stripping and skin scraping.

In certain embodiments, the method of detecting expression of genes in the skin involves applying an adhesive tape to a target area of the skin in a manner sufficient to isolate an epidermal sample adhering to the adhesive tape, wherein the epidermal sample comprises a gene product. The gene products in the epidermal sample are then detected. In some embodiments, gene products are applied to a microarray to detect the gene products. In some embodiments, the gene product is isolated from the epidermal sample. In some embodiments, the gene product is a nucleic acid molecule, such as an RNA or a DNA molecule. In some embodiments, nucleic acid is amplified. In some embodiments, the gene product is a polypeptide.

Accordingly, in one embodiment, the present invention employs a non-invasive tape stripping technology to obtain samples of suspicious lesions. As such, DNA microarray assays are used to create a non-invasive diagnostic for melanoma and/or distinguishing melanoma from solar lentigo. Tape-stripping removes superficial cells from the surface of the skin as well as adnexal cells. Small amounts of nucleic acid molecules isolated from tape-stripped cells can be amplified and used for microarray analyses and quantitative PCR. In addition, proteins obtained from the lysed cells may be quantitated for diagnosis of disease. Consequently, tape-stripping is a non-invasive diagnostic method, which does not interfere with subsequent histological analyses, thereby bypassing a major limitation to current expression profiling studies on melanoma. While tape stripping will primarily sample superficial cells from the epidermis, this method holds great promise in the diagnoses and prognosis prediction in pigmented lesions for the following reasons: First, in contrast to benign nevi, in many melanomas the pigmented cells migrate into the epidermis and/or adnexa. Consequently, this feature may help differentiate benign pigmented lesions from melanomas based on tape stripping. Second, there are changes in the dermis and epidermis adjacent to melanoma. The epidermal hyperplasia overlying melanoma seems to correlate with both angiogenesis and metastatic potential; these changes are expected to be sampled with the tape stripping method. Finally, some advanced melanomas do reach the surface of the skin and melanoma cancer cells would be sampled directly by the tape stripping. In addition tape stripping is useful in the care of patients with multiple pigmented lesions where it is unpractical to biopsy each and every lesion. Accordingly, the present invention demonstrates that stratum corneum RNA, harvested by tape stripping with Epidermal Genetic Information Retrieval (EGIR) (see U.S. Pat. No. 6,949,338, incorporated herein by reference), can be used to distinguish melanoma from dysplastic nevi in suspicious pigmented lesions.

As indicated, the tape stripping methods provided herein typically involve applying an adhesive tape to the skin of a subject and removing the adhesive tape from the skin of the subject one or more times. In certain examples, the adhesive tape is applied to the skin and removed from the skin about one to ten times. Alternatively, about ten adhesive tapes can be sequentially applied to the skin and removed from the skin. These adhesive tapes are then combined for further analysis. Accordingly, an adhesive tape can be applied to and removed from a target site 10, 9, 8, 7, 6, 5, 4, 3, 2, or 1 time, and/or 10, 9, 8, 7, 6, 5, 4, 3, 2, or 1 adhesive tape can be applied to and removed from the target site. In one illustrative example, the adhesive tape is applied to the skin between about one and eight times, in another example, between one and five times, and in another illustrative example the tape is applied and removed from the skin four times.

In some embodiments, the adhesive tape is pliable. In some embodiments, the adhesive tape comprises a non-polar polymer adhesive. In some embodiments, the adhesive tape comprises a rubber-based adhesive.

In certain instances, non-polar, pliable adhesive tapes, including plastic-based adhesive tapes, are effective for obtaining epidermal samples from the skin. In certain instances, non-polar, pliable adhesive tapes, including plastic-based adhesive tapes, are more effective for obtaining epidermal samples from the skin than other types of adhesive tapes. Accordingly, in some embodiments, a non-polar, pliable adhesive tapes are applied in as few as 10 or less tape strippings, such as 9, 8, 7, 6, 5, 4, 3, 2, or 1 tape stripping, to obtain a sample. In some embodiments, the tape strippings method is employed to isolate a gene product from the epidermis of skin for gene expression analysis.

In some embodiments, the rubber based adhesive is a synthetic rubber-based adhesive. In some embodiments, the rubber based adhesive has high peel, high shear, and high tack. For example, in some embodiments, the rubber based adhesive has a peak force tack that is at least 25%, 50%, or 100% greater than the peak force tack of an acrylic-based tape such as D-squame™. D-squame™ has been found to have a peak force of 2 Newtons. In some embodiments, the peak force of the rubber based adhesive used for methods provided herein is about 4 Newtons or greater. In some embodiments, the rubber based adhesive has adhesion that is greater than 2 times, 5 times, or times that of acrylic based tape. D-squame™ has been found to have adhesion of 0.0006 Newton meters. In some embodiments, the rubber based tape provided herein has an adhesion of about 0.01 Newton meters using a texture analyzer. In some embodiments, the adhesive used in the methods provided herein has higher peel, shear and tack compared to other rubber adhesives, such as those used for medical application and Duct tape.

In some embodiments, the rubber-based adhesive is more hydrophobic than acrylic adhesives. In some embodiments, the rubber based adhesive is inert to biomolecules and to chemicals used to isolate biomolecules, including proteins and nucleic acids, such as DNA and RNA. In some embodiments, the rubber-based adhesive is relatively soft compared to other tapes such as D-squame™.

In some embodiments, the rubber-based adhesive is on a support, such as a film, that makes the tape pliable and flexible. In certain aspects, the tape is soft and pliable. As used herein, “pliable” tape is tape that is easily bent or shaped. As used herein, “soft and pliable” tape is tape that is easily bent or shaped and yields readily to pressure or weight. In some embodiments, the film is made of any of many possible polymers, provided that the tape is pliable and can be used with a rubber adhesive. In some embodiments, the film is a polyurethane film such as skin harvesting tape (Product No. 90068) available from Adhesives Research, Inc (Glen Rock, Pa.). In some embodiments, the thickness is varied provided that the tape remains pliable. For example, in some embodiments, the tape is about 0.5 mm to about 10 mm in thickness, such as about 1.0 to about 5.0 mm in thickness. In one example, the tape contains a rubber adhesive on a 3.0 mm polyurethane film.

Virtually any size and/or shape of adhesive tape and target skin site size and shape can be used and analyzed, respectively, by the methods of the present invention. For example, adhesive tape can be fabricated into circular discs of diameter between 10 millimeters and 100 millimeters, for example between 15 and 25 millimeters in diameter. The adhesive tape can have a surface area of between about 50 mm² and 1000 mm², between about 100 mm² to 500 mm² or about 250 mm².

Described herein, in certain embodiments, are adhesive tapes comprising an epidermal sample of a suspected melanoma lesion that comprises a gene product expressed by one or more genes in any of ACTB, ACTN4, B2M, LINC00518 (also known as C6orf218 and/or MGC40222), CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, or VIM, wherein the epidermal sample is of a sufficient quantity to allow determination of the relative amount of gene product present in the epidermal sample.

In another embodiment, the sample is obtained by means of an invasive procedure, such as biopsy. Biopsies may be taken instead of or after tape stripping and are subjected to standard histopathologic analysis. Analysis of biopsy samples taken simultaneously with tape stripping samples may then be correlated with the data generated from one or more of analysis of selected lesion RNA samples by DNA microarray, correlation of gene expression data with histopathology, and creation of a candidate expression classifier for diagnosis of melanoma.

As used herein, “biopsy” refers to the removal of cells or tissues for analysis. There are many different types of biopsy procedures known in the art. The most common types include: (1) incisional biopsy, in which only a sample of tissue is removed; (2) excisional biopsy, in which an entire lump or suspicious area is removed; and (3) needle biopsy, in which a sample of tissue or fluid is removed with a needle. When a wide needle is used, the procedure is called a core biopsy. When a thin needle is used, the procedure is called a fine-needle aspiration biopsy. Other types of biopsy procedures include, but are not limited to, shave biopsy, punch biopsy, curettage biopsy, and in situ biopsy. In another embodiment, the skin sample is obtained by scraping the skin with an instrument to remove one or more nucleic acid molecules from the skin.

The skin sample obtained using the tape stripping method includes, epidermal cells including cells comprising adnexal structures. In certain illustrative examples, the sample includes predominantly epidermal cells, or even exclusively epidermal cells. The epidermis consists predominantly of keratinocytes (>90%), which differentiate from the basal layer, moving outward through various layers having decreasing levels of cellular organization, to become the cornified cells of the stratum corneum layer. Renewal of the epidermis occurs every 20-30 days in uninvolved skin. Other cell types present in the epidermis include melanocytes, Langerhans cells, and Merkel cells. As illustrated in the Examples herein, the tape stripping method of the present invention is particularly effective at isolating epidermal samples.

In another aspect, the methods of the present invention involve in situ analysis of the skin lesion for characterization thereof. For in situ methods, nucleic acid molecules do not need to be isolated from the subject prior to analysis. In one embodiment, detectably labeled probes are contacted with a cell or tissue of a subject for visual detection of expressed RNA to characterize the skin lesion.

In some embodiments, the methods of the present invention can be used with less than 100,000 skin cells. In some embodiments, the methods of the present invention are practiced with less than 75,000 skin cells. In some embodiments, the methods of the present invention are practiced with less than 50,000 skin cells. In some embodiments, the methods of the present invention are practiced with less than 25,000 skin cells. In some embodiments, the methods of the present invention are practiced with less than 10,000 skin cells. In some embodiments, the methods of the present invention are practiced with less than 5,000 skin cells. In some embodiments, the methods of the present invention are practiced with less than 1,000 skin cells. In some embodiments, the methods of the present invention are practiced with less than 900 skin cells. In some embodiments, the methods of the present invention are practiced with less than 800 skin cells. In some embodiments, the methods of the present invention are practiced with less than 700 skin cells. In some embodiments, the methods of the present invention are practiced with less than 600 skin cells. In some embodiments, the methods of the present invention are practiced with less than 500 skin cells. In some embodiments, the methods of the present invention are practiced with less than 400 skin cells. In some embodiments, the methods of the present invention are practiced with less than 300 skin cells. In some embodiments, the methods of the present invention are practiced with less than 200 skin cells. In some embodiments, the methods of the present invention are practiced with less than 100 skin cells.

The target nucleic acid that is ultimately analyzed can be prepared synthetically (in the case of control sequences), but typically is purified from the skin sample and subjected to one or more preparative steps. The nucleic acid may be purified to remove or diminish one or more undesired components from the biological sample or to concentrate it. Conversely, where the nucleic acid is too concentrated for the particular assay, it may be diluted.

### Methods for Isolation of a Gene Product

In certain embodiments, the gene products are isolated from the epidermal samples. In some embodiments, the cells of the epidermal samples are lysed. In some embodiments, the cells of the epidermal samples are lysed and the gene products are isolated from lysed cells.

In certain embodiments, nucleic acid molecules are isolated from the lysed cells and cellular material by any number of means well known to those skilled in the art. For example, in some embodiments, any of a number of commercial products available for isolating nucleic acid molecules, including, but not limited to, RNeasy™ (Qiagen, Valencia, Calif.) and TriReagent™ (Molecular Research Center, Inc, Cincinnati, Ohio), is used. In some embodiments, the isolated nucleic acid molecules are then tested or assayed for particular nucleic acid sequences. In some embodiments, the isolated nucleic acid molecules are then tested or assayed for a nucleic acid sequence that represents a gene product of any of the genes ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, or VIM. Methods of detecting a target nucleic acid molecule within a nucleic acid sample are well known in the art. In some embodiments, detecting a target nucleic acid molecule involves a hybridization technique such as a microarray analysis or sequence specific nucleic acid amplification. In some embodiments, detecting a target nucleic acid molecule involves sequencing.

In some embodiments, one or more of the nucleic acid molecules in a sample provided herein, such as a as an epidermal sample, is amplified before or after they are isolated and/or detected. The term “amplified” refers to the process of making multiple copies of the nucleic acid from a single nucleic acid molecule. In some embodiments, the amplification of nucleic acid molecules is carried out in vitro by biochemical processes known to those of skill in the art. In some embodiments, the amplification agent is any compound or system that will function to accomplish the synthesis of primer extension products, including enzymes. It will be recognized that various amplification methodologies can be utilized to increase the copy number of a target nucleic acid in the nucleic acid samples obtained using the methods provided herein, before and after detection. Suitable enzymes for this purpose include, for example, E. coli DNA polymerase I, Taq polymerase, Klenow fragment of E. coli DNA polymerase I, T4 DNA polymerase, other available DNA polymerases, T4 or T7 RNA polymerase, polymerase muteins, reverse transcriptase, ligase, and other enzymes, including heat-stable enzymes (i.e., those enzymes that perform primer extension after being subjected to temperatures sufficiently elevated to cause denaturation or those using an RNA polymerase promoter to make a RNA from a DNA template, i.e. linearly amplified aRNA).

Suitable enzymes will facilitate incorporation of nucleotides in the proper manner to form the primer extension products that are complementary to each nucleotide strand. Generally, the synthesis will be initiated at the 3′-end of each primer and proceed in the 5′-direction along the template strand, until synthesis terminates, producing molecules of different lengths. There can be amplification agents, however, that initiate synthesis at the 5′-end and proceed in the other direction, using the same process as described above. In any event, the method provided herein is not to be limited to the amplification methods described herein since it will be understood that virtually any amplification method can be used.

In some embodiments, polymerase chain reaction (PCR) is employed for nucleic acid amplification (described, e.g., in U.S. Pat. Nos. 4,683,202 and 4,683,195). It will be understood that optimal conditions for a PCR reaction can be identified using known techniques. In one illustrative example, RNA is amplified using the MessageAmp™aRNA kit (as disclosed in the Examples herein).

In some embodiments, the primers for use in amplifying the polynucleotides of the invention are prepared using any suitable method, such as conventional phosphotriester and phosphodiester methods or automated embodiments thereof so long as the primers are capable of hybridizing to the polynucleotides of interest. One method for synthesizing oligonucleotides on a modified solid support is described in U.S. Pat. No. 4,458,066. The exact length of primer will depend on many factors, including temperature, buffer, and nucleotide composition. The primer must prime the synthesis of extension products in the presence of the inducing agent for amplification.

Primers used according to the method of the invention are complementary to each strand of nucleotide sequence to be amplified. The term “complementary” means that the primers must hybridize with their respective strands under conditions, which allow the agent for polymerization to function. In other words, the primers that are complementary to the flanking sequences hybridize with the flanking sequences and permit amplification of the nucleotide sequence. The 3′ terminus of the primer that is extended can have perfect base paired complementarity with the complementary flanking strand, or can hybridize to the flanking sequences under high stringency conditions.

In some embodiments, upon isolation and optional amplification, expression of one or more genes is analyzed. Analyzing expression includes any qualitative or quantitative method for detecting expression of a gene, many of which are known in the art. Non-limiting methods for analyzing polynucleotides and polypeptides are discussed below. The methods of analyzing expression of the present invention can utilize a biochip, or other miniature high-throughput technology, for detecting expression of two or more genes.

In some embodiments, the methods provided involve isolation of RNA, including messenger RNA (mRNA), from a skin sample. In some embodiments, RNA is single stranded or double stranded. In some embodiments, enzymes and conditions optimal for reverse transcribing the template to DNA well known in the art are used. In some embodiments, the RNA is amplified to form amplified RNA. In some embodiments, the RNA is subjected to RNAse protection assays. In some embodiments, a DNA-RNA hybrid that contains one strand of each is used. In some embodiments, a mixture of polynucleotides is employed, or the polynucleotides produced in a previous amplification reaction, using the same or different primers are used. In certain examples, a nucleic acid to be analyzed is amplified after it is isolated. It is not necessary that the sequence to be amplified be present initially in a pure form; it may be a minor fraction of a complex mixture.

### Detection Methods

In some embodiments, a microarray is employed for detection of an expressed gene product. The manufacture and use of biochips such as those involving microarrays, also known as bioarrays, are known in the art. (For reviews of Biochips and microarrays see, e.g., Kallioniemi O. P., “Biochip technologies in cancer research,” Ann Med, March; 33(2):142 7 (2001); and Rudert F., “Genomics and proteomics tools for the clinic,” Curr Opin. Mol. Ther., December; 2(6):633 42 (2000)) Furthermore, a number of biochips for expression analysis are commercially available (See e.g., microarrays available from Sigma-Genosys (The Woodlands, Tex.); Affymetrix (Santa Clara, Calif.), and Full Moon Biosystems (Sunnyvale, Calif.)). In some embodiments, such microarrays are analyzed using blotting techniques similar to those discussed below for conventional techniques of detecting polynucleotides and polypeptides. In some embodiments, detailed protocols for hybridization conditions are available through manufacturers of microarrays. In some embodiments, a microarray provides for the detection and analysis of at least 10, 20, 25, 50, 100, 200, 250, 500, 750, 1000, 2500, 5000, 7500, 10,000, 12,500, 25,000, 50,0000, or 100,000 genes.

In some embodiments, for microarray expression analysis, isolated RNA is amplified. In some embodiments, the amplified RNA is then used for hybridization to sequence specific nucleic acid probes on a biochip. In some embodiments, amplification is performed using a commercially available kit, such as MessageAMp™ RNA kit (Ambion Inc.). In some embodiments, isolated RNA is labeled before contacting the biochip such that binding to the target array can be detected using streptavidin. In some embodiments, isolated RNA is labeled with a detectable moiety, such as, but not limited to, a fluorescent moiety, a dye, or a ligand, such as biotin. In some embodiments, the nucleic acid probes of the microarray bind specifically to one or more of the gene products of the genes selected from ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, and VIM. In some embodiments, the nucleic acid probes of the microarray bind specifically to one or more of the gene products of the genes selected from LINC00518, TRIB2, KIT, SDCBP, TYR, NAMPT, ACTN4, EDNRB, GPM6B, CNN2, MCOLN3, PRAME, TMEM80, TTC3, and CMIP. In some embodiments, the nucleic acid probes of the microarray bind specifically to one or more of the gene products of the genes selected from ACTN4, LINC00518, CMIP, TTC3, TMEM80, HLA-C, and PRAME.

In some embodiments, hybridization of amplified nucleic acids to probes on a microarray is typically performed under stringent hybridization conditions. Conditions for hybridization reactions are well known in the art and are available from microarray suppliers. For example, in some embodiments, hybridization of a nucleic acid molecule with probes found on a microarray is performed under moderately stringent or highly stringent physiological conditions, as are known in the art. For example, in some embodiments, hybridization on a microarray is performed according to manufacturer's (Affymetrix) instructions. For example, in some embodiments, hybridization is performed for 16 hours at 45° C. in a hybridization buffer, such as 100 mM MES, 1 M [Na⁺], 20 mM EDTA, 0.01% Tween 20. In some embodiments, washes are performed in a low stringency buffer ((6×SSPE, 0.01% Tween 20) at 25° C. followed by a high stringency buffer (100 mM MES, 0.1M [Na⁺], 0.01% Tween 20) at 5° C. In some embodiments, washes are performed using progressively higher stringency conditions: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). In some embodiments, washing is carried out using only one of these conditions, for example, high stringency conditions. In some embodiments, washing is carried out using each of the conditions. In some embodiments, washing is carried out using each of the conditions, for 10 to 15 minutes each, in the order listed above, optionally repeating any or all of the steps listed.

In some embodiments, other microfluidic devices and methods for analyzing gene expression, including those in which more than one gene can be analyzed simultaneously and those involving high-throughput technologies, are used for the methods provided herein.

Quantitative measurement of expression levels using bioarrays is also known in the art, and typically involves a modified version of a traditional method for measuring expression as described herein. For example, such quantitation can be performed by measuring a phosphor image of a radioactive-labeled probe binding to a spot of a microarray, using a phosphohor imager and imaging software.

Many statistical techniques are known in the art, which can be used to determine whether a statistically significant difference in expression is observed at a 90% or preferably a 95% confidence level.

In some embodiments, RNAse protection assays is used where RNA is the polynucleotide to be detected in the method. In this procedure, a labeled antisense RNA probe is hybridized to the complementary polynucleotide in the sample. The remaining unhybridized single-stranded probe is degraded by ribonuclease treatment. The hybridized, double stranded probe is protected from RNAse digestion. After an appropriate time, the products of the digestion reaction are collected and analyzed on a gel (see for example Ausubel et al., CURRENT PROTOCOLS IN MOLECULAR BIOLOGY, section 4.7.1 (1987)). As used herein, “RNA probe” refers to a ribonucleotide capable of hybridizing to RNA in a sample of interest. Those skilled in the art will be able to identify and modify the RNAse protection assay specific to the polynucleotide to be measured, for example, probe specificity can be altered, hybridization temperatures, quantity of nucleic acid etc. Additionally, a number of commercial kits are available, for example, RiboQuant™ Multi-Probe RNAse Protection Assay System (Pharmingen, Inc., San Diego, Calif.).

In another embodiment, a nucleic acid in the sample is analyzed by a blotting procedure, typically a Northern blot procedure. For blotting procedures polynucleotides are separated on a gel and then probed with a complementary polynucleotide to the sequence of interest. For example, RNA is separated on a gel transferred to nitrocellulose and probed with complementary DNA to one of the genes disclosed herein. In some embodiments, complementary probe is labeled such as radioactively or chemically.

In some embodiments, detection of a nucleic acid includes size fractionating the nucleic acid. Methods of size fractionating nucleic acids are well known to those of skill in the art, such as by gel electrophoresis, including polyacrylamide gel electrophoresis (PAGE). For example, in some embodiments, the gel is a denaturing 7 M or 8 M urea-polyacrylamide-formamide gel. In some embodiments, size fractionating the nucleic acid is accomplished by chromatographic methods known to those of skill in the art.

In some embodiments, the detection of nucleic acids is performed by using radioactively labeled probes. In some embodiments, any radioactive label is employed which provides an adequate signal. Other labels include ligands, colored dyes, and fluorescent molecules, which, in some embodiments, serve as a specific binding pair member for a labeled ligand, and the like. The labeled preparations are used to probe for a nucleic acid by the Southern or Northern hybridization techniques, for example. Nucleotides obtained from samples are transferred to filters that bind polynucleotides. After exposure to the labeled polynucleotide probe, which will hybridize to nucleotide fragments containing target nucleic acid sequences, the binding of the radioactive probe to target nucleic acid fragments is identified by autoradiography (see Genetic Engineering, 1 ed. Robert Williamson, Academic Press (1981), pp. 72 81). The particular hybridization technique is not essential to the performance of the method provided. Hybridization techniques are well known or easily ascertained by one of ordinary skill in the art. As improvements are made in hybridization techniques, they can readily be applied in the method of the invention.

In some embodiments, probes according for use in the methods provided selectively hybridize to a target gene. In some embodiments, the probes are spotted on a bioarray using methods known in the art. As used herein, the term “selective hybridization” or “selectively hybridize,” refers to hybridization under moderately stringent or highly stringent conditions such that a nucleotide sequence preferentially associates with a selected nucleotide sequence over unrelated nucleotide sequences to a large enough extent to be useful in detecting expression of a skin marker. It will be recognized that some amount of non-specific hybridization is unavoidable, but is acceptable provide that hybridization to a target nucleotide sequence is sufficiently selective such that it can be distinguished over the non-specific cross-hybridization, for example, at least about 2-fold more selective, generally at least about 3-fold more selective, usually at least about 5-fold more selective, and particularly at least about 10-fold more selective, as determined, for example, by an amount of labeled oligonucleotide that binds to target nucleic acid molecule as compared to a nucleic acid molecule other than the target molecule, particularly a substantially similar (i.e., homologous) nucleic acid molecule other than the target nucleic acid molecule.

In some embodiments, conditions that allow for selective hybridization are determined empirically, or estimated based, for example, on the relative GC:AT content of the hybridizing oligonucleotide and the sequence to which it is to hybridize, the length of the hybridizing oligonucleotide, and the number, if any, of mismatches between the oligonucleotide and sequence to which it is to hybridize (see, for example, Sambrook et al., “Molecular Cloning: A laboratory manual (Cold Spring Harbor Laboratory Press 1989)). An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42EC (moderate stringency conditions); and 0.1×SSC at about 68EC (high stringency conditions). In some embodiments, washing is carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

In some embodiments, a method for detecting one or more gene products employs the detection of a polypeptide product of one of these genes. For example, in some embodiments, polypeptide products of one of the genes disclosed herein, is analyzed. In some embodiments, the levels of such gene products are indicative of melanoma when compared to a normal or standard polypeptide profiles in a similar tissue. In this regard, the sample, as described herein, is used as a source to isolate polypeptides. For example, in some embodiments, following skin stripping, using the methods described above, cells isolated from the stratum corneum are lysed by any number of means, and polypeptides obtained from the cells. In some embodiments, these polypeptides are quantified using methods known to those of skill in the art, for example by protein microarrays, or ELISA analysis.

In another embodiment, provided are methods for obtaining gene expression data from amplified nucleic acids that compensates for variability in amplification reactions. In this method, relative expression of a target nucleic acid molecule and a control nucleic acid molecule is compared to obtain relevant expression data. Accordingly, in certain embodiments, a ΔCt value is determined in order to identify gene expression changes. In some embodiments, this value and method is used to identify differential gene expression in any tissue, including the tape stripped skin samples provided herein. Such method is especially useful, where it is relatively difficult to obtain sufficient RNA from a control sample.

The Cₜ value is the experimentally determined number of amplification (e.g. PCR) cycles required to achieve a threshold signal level (statistically significant increase in signal level (e.g. fluorescence) over background) for mRNAₓ and a control mRNA (Gibson, Heid et al. 1996; Heid, Stevens et al. 1996). The Ct values are typically determined using a target nucleic acid (e.g. mRNAx) primer and probe set, and a control mRNA primer and probe set. A Δ Cₜ value is calculated by calculating a difference in the number of amplification cycles required to reach a threshold signal level between the target nucleic acid molecule and the control nucleic acid molecule. A difference in the Δ Cₜ value at a target area versus another area of a subject's skin, such as a normal area, or an unaffected area, is indicative of a change in gene expression of the target nucleic acid molecule at the target area. Using this value, altered expression is detected by comparing expression of the target nucleic acid molecule with expression of a control nucleic acid molecule. The Δ Cₜ value is useful for characterizing the physiologic state of the epidermis without reference to a calibration site. Such methods provide the advantage that it is not necessary to obtain a nucleic acid sample from a control site, where it may be difficult to obtain sufficient nucleic acid molecules.

Accordingly, provided herein is a method for detecting a change in gene expression, including: applying a first adhesive tape to a target area of skin, such as a pigmented skin lesion, and a second adhesive tape to an unaffected area of the skin, in a manner sufficient to isolate an epidermal sample adhering to the first adhesive tape and the second adhesive tape, wherein the epidermal samples comprise nucleic acid molecules; and for each of the target area sample and the normal area sample, amplifying a target nucleic acid molecule and a control nucleic acid molecule. For each of the target area sample and the normal area sample, a target nucleic acid molecule and a control nucleic acid molecule are amplified and identifying, and Δ Cₜ value by calculating a difference in the number of amplification cycles required to reach a threshold signal level between the target nucleic acid molecule and a control nucleic acid molecule, wherein a difference in the Δ Cₜ value at the target area versus the normal area is indicative of a change in gene expression of the target nucleic acid molecule at the target area. The Ct values are typically determined in the same amplification experiment (e.g. using separate reaction wells on the same multi-well reaction plate) using similar reaction conditions to other reactions.

In some embodiments, the method for detecting a change in gene expression is used along with the other embodiments provided herein to identify changes in gene expression. For example, in some embodiments, the method is used to diagnose a pigmented skin lesion as being melanoma. In certain aspects, the method is used to detect a change in expression for any of the genes selected from ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, and VIM to assist in a characterization of a skin lesion. In certain aspects, the method is used to detect a change in expression for any of the genes selected from LINC00518, TRIB2, KIT, SDCBP, TYR, NAMPT, ACTN4, EDNRB, GPM6B, CNN2, MCOLN3, PRAME, TMEM80, TTC3, and CMIP to assist in a characterization of a skin lesion. In certain aspects, the method is used to detect a change in expression for any of the genes selected from ACTN4, LINC00518, CMIP, TTC3, TMEM80, HLA-C, and PRAME to assist in a characterization of a skin lesion. In certain aspects, the method is used to detect a change in expression in ACTN4, LINC00518, CMIP, TTC3, TMEM80, HLA-C, and PRAME to assist in a characterization of a skin lesion. In certain aspects, the method is used to detect a change in expression in LINC00518 to assist in a characterization of a skin lesion. In certain aspects, the method is used to detect a change in expression in LINC00518 and CMIP to assist in a characterization of a skin lesion. In certain aspects, the method is used to detect a change in expression in LINC00518 and TTC3 to assist in a characterization of a skin lesion. In certain aspects, the method is used to detect a change in expression in LINC00518 and TMEM80 to assist in a characterization of a skin lesion. In certain aspects, the method is used to detect a change in expression in LINC00518 and HLA-C to assist in a characterization of a skin lesion. In certain aspects, the method is used to detect a change in expression in LINC00518 and PRAME to assist in a characterization of a skin lesion. In certain aspects, the method is used to detect a change in expression in LINC00518 and NAMPT to assist in a characterization of a skin lesion. In certain aspects, the method is used to detect a change in expression in LINC00518, ACTN4 and CMIP to assist in a characterization of a skin lesion. In certain aspects, the method is used to detect a change in expression in LINC00518 and TRIB2 to assist in a characterization of a skin lesion. In certain aspects, the method is used to detect a change in expression in LINC00518 and GPM6B to assist in a characterization of a skin lesion. In certain aspects, the method is used to detect a change in expression in TRIB2 and NAMPT to assist in a characterization of a skin lesion. In certain aspects, the method is used to detect a change in expression in TRIB2 and KIT to assist in a characterization of a skin lesion. In certain aspects, the method is used to detect a change in expression in TRIB2 and ACTN4 to assist in a characterization of a skin lesion.

### Methods for Analyzing a Nucleic Acid Molecule

Described herein, in some embodiments, is a method for characterizing a skin lesion in a subject, comprising analyzing a nucleic acid molecule from one or more genes comprising: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, or VIM, in a sample of the skin lesion, thereby producing a characterization of the skin lesion. In some embodiments, two or more genes selected from ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, and VIM are analyzed. In some embodiments, LINC00518 is analyzed. In some embodiments, the genes are selected from LINC00518, TRIB2, KIT, SDCBP, TYR, NAMPT, ACTN4, EDNRB, GPM6B, CNN2, MCOLN3, PRAME, TMEM80, TTC3, and CMIP, and combinations thereof. In some embodiments, the genes are selected from LINC00518, CMIP, ACTN4, TMEM80 and NAMPT and combinations thereof. In some embodiments, the one or more genes are selected from LINC00518 and CMIP, and combinations thereof. In some embodiments, the combination of LINC00518 and CMIP is analyzed. In some embodiments, the one or more genes are selected from LINC00518 and TMEM80, and combinations thereof. In some embodiments, the combination of LINC00518 and TMEM80 is analyzed. In some embodiments, the one of more genes are selected from LINC00518 and ACTN4, and combinations thereof. In some embodiments, the combination of LINC00518 and ACTN4 is analyzed. In some embodiments, the combination of LINC00518 and NAMPT are analyzed. In some embodiments, the two or more genes comprise LINC00518 and TTC3, and combinations thereof. In some embodiments, the combination of LINC00518 and TTC3 are analyzed. In some embodiments, the two or more genes comprise LINC00518 and HLA-C, and combinations thereof. In some embodiments, the combination of LINC00518 and HLA-C are analyzed. In some embodiments, the two or more genes comprise LINC00518 and PRAME, and combinations thereof. In some embodiments, the combination of LINC00518 and PRAME are analyzed. In some embodiments, the two or more genes comprise ACTN4, LINC00518 and PRAME, and combinations thereof. In some embodiments, the combination of ACTN4, LINC00518 and PRAME are analyzed. In some embodiments, the two or more genes comprise LINC00518 and TRIB2, and combinations thereof. In some embodiments, the two or more genes comprise LINC00518 and TRIB2, and combinations thereof. In some embodiments, the two or more genes comprise LINC00518 and GPM6B, and combinations thereof. In some embodiments, the combination of LINC00518 and GPM6B is analyzed. In some embodiments, the two or more genes comprise TRIB2 and NAMPT, and combinations thereof. In some embodiments, the combination of TRIB2 and NAMPT is analyzed. In some embodiments, the two or more genes comprise TRIB2 and KIT, and combinations thereof. In some embodiments, the combination of TRIB2 and KIT is analyzed. In some embodiments, the combination of TRIB2 and ACTN4 is analyzed. In some embodiments, the combination of TRIB2 and ACTN4 is analyzed.

In some embodiments, a two-gene classifier for the diagnosis of melanoma is analyzed (e.g., the expression of two genes is analyzed to calculate the classifier value). In some embodiments, the two-gene classifier has a greater than about 75% or 90% confidence interval for distinguishing non-melanoma from melanoma. In some embodiments, the two-gene classifier has a greater than about 75% or 90% confidence interval for distinguishing melanoma in situ from invasive melanoma. In some embodiments, the two-gene classifier has a greater than about 75% or 90% confidence interval for distinguishing non-melanoma, melanoma in situ, and invasive melanoma. In some embodiments, the two-gene classifier is LINC00518 and CMIP. In some embodiments, the two-gene classifier is LINC00518 and TMEM80. In some embodiments, the two-gene classifier is LINC00518 and ACTN4. In some embodiments, the two-gene classifier is LINC00518 and NAMPT. In some embodiments, the two-gene classifier is LINC00518 and TTC3. In some embodiments, the two-gene classifier is LINC00518 and HLA-C. In some embodiments, the two-gene classifier is LINC00518 and PRAME. In some embodiments, the two-gene classifier is LINC00518 and TRIB2. In some embodiments, the two-gene classifier is LINC00518 and GPM6B. In some embodiments, the two-gene classifier is TRIB2 and NAMPT. In some embodiments, the two-gene classifier is TRIB2 and KIT. In some embodiments, the two-gene classifier is TRIB2 and ACTN4. In further or additional embodiments, the two-gene classifier comprises LINC00518 or CMIP. In some embodiments, the two-gene classifier comprises LINC00518. In some embodiments, the two-gene classifier comprises CMIP.

In some embodiments, a two-gene classifier for the diagnosis of melanoma is analyzed (e.g., the expression of two genes is analyzed to calculate the classifier value). In some embodiments, the two-gene classifier has a greater than about 95% confidence interval for distinguishing non-melanoma from melanoma. In some embodiments, the two-gene classifier has a greater than about 95% confidence interval for distinguishing melanoma in situ from invasive melanoma. In some embodiments, the two-gene classifier has a greater than about 95% confidence interval for distinguishing non-melanoma, melanoma in situ, and invasive melanoma. In some embodiments, the two-gene classifier is LINC00518 and CMIP. In some embodiments, the two-gene classifier is LINC00518 and TMEM80. In some embodiments, the two-gene classifier is LINC00518 and ACTN4. In some embodiments, the two-gene classifier is LINC00518 and NAMPT. In some embodiments, the two-gene classifier is LINC00518 and TTC3. In some embodiments, the two-gene classifier is LINC00518 and HLA-C. In some embodiments, the two-gene classifier is LINC00518 and PRAME. In some embodiments, the two-gene classifier is LINC00518 and TRIB2. In some embodiments, the two-gene classifier is LINC00518 and GPM6B. In some embodiments, the two-gene classifier is TRIB2 and NAMPT. In some embodiments, the two-gene classifier is TRIB2 and KIT. In some embodiments, the two-gene classifier is TRIB2 and ACTN4. In further or additional embodiments, the two-gene classifier comprises LINC00518 or CMIP. In some embodiments, the two-gene classifier comprises LINC00518. In some embodiments, the two-gene classifier comprises CMIP.

In some embodiments, a two-gene classifier for the diagnosis of melanoma is analyzed (e.g., the expression of two genes is analyzed to calculate the classifier value). In some embodiments, the two-gene classifier has a greater than about 97% confidence interval for distinguishing non-melanoma from melanoma. In some embodiments, the two-gene classifier has a greater than about 97% confidence interval for distinguishing melanoma in situ from invasive melanoma. In some embodiments, the two-gene classifier has a greater than about 97% confidence interval for distinguishing non-melanoma, melanoma in situ, and invasive melanoma. In some embodiments, the two-gene classifier is LINC00518 and CMIP. In some embodiments, the two-gene classifier is LINC00518 and TMEM80. In some embodiments, the two-gene classifier is LINC00518 and ACTN4. In some embodiments, the two-gene classifier is LINC00518 and NAMPT. In some embodiments, the two-gene classifier is LINC00518 and TTC3. In some embodiments, the two-gene classifier is LINC00518 and HLA-C. In some embodiments, the two-gene classifier is LINC00518 and PRAME. In some embodiments, the two-gene classifier is LINC00518 and TRIB2. In some embodiments, the two-gene classifier is LINC00518 and GPM6B. In some embodiments, the two-gene classifier is TRIB2 and NAMPT. In some embodiments, the two-gene classifier is TRIB2 and KIT. In some embodiments, the two-gene classifier is TRIB2 and ACTN4. In further or additional embodiments, the two-gene classifier comprises LINC00518 or CMIP. In some embodiments, the two-gene classifier comprises LINC00518. In some embodiments, the two-gene classifier comprises CMIP.

In some embodiments, a two-gene classifier for the diagnosis of melanoma is analyzed (e.g., the expression of two genes is analyzed to calculate the classifier value). In some embodiments, the two-gene classifier has a greater than about 99% confidence interval for distinguishing non-melanoma from melanoma. In some embodiments, the two-gene classifier has a greater than about 99% confidence interval for distinguishing melanoma in situ from invasive melanoma. In some embodiments, the two-gene classifier has a greater than about 99% confidence interval for distinguishing non-melanoma, melanoma in situ, and invasive melanoma. In some embodiments, the two-gene classifier is LINC00518 and CMIP. In some embodiments, the two-gene classifier is LINC00518 and TMEM80. In some embodiments, the two-gene classifier is LINC00518 and ACTN4. In some embodiments, the two-gene classifier is LINC00518 and NAMPT. In some embodiments, the two-gene classifier is LINC00518 and TTC3. In some embodiments, the two-gene classifier is LINC00518 and HLA-C. In some embodiments, the two-gene classifier is LINC00518 and PRAME. In some embodiments, the two-gene classifier is LINC00518 and TRIB2. In some embodiments, the two-gene classifier is LINC00518 and GPM6B. In some embodiments, the two-gene classifier is TRIB2 and NAMPT. In some embodiments, the two-gene classifier is TRIB2 and KIT. In some embodiments, the two-gene classifier is TRIB2 and ACTN4. In further or additional embodiments, the two-gene classifier comprises LINC00518 or CMIP. In some embodiments, the two-gene classifier comprises LINC00518. In some embodiments, the two-gene classifier comprises CMIP.

In some embodiments, a three-gene classifier for the diagnosis of melanoma is analyzed (e.g., the expression of three genes is analyzed to calculate the classifier value). In some embodiments, the three-gene classifier has a greater than about 75%, 90%, 95%, 97%, or 99% confidence interval for distinguishing non-melanoma from melanoma. In some embodiments, the three-gene classifier has a greater than about 75%, 90%, 95%, 97%, or 99% confidence interval for distinguishing melanoma in situ from invasive melanoma. In some embodiments, the three-gene classifier has a greater than about 75%, 90%, 95%, 97%, or 99% confidence interval for distinguishing non-melanoma, melanoma in situ, and invasive melanoma. In some embodiments, the three-gene classifier is ACTN4, LINC00518 and PRAME.

In some embodiments, a four-gene classifier for the diagnosis of melanoma is analyzed (e.g., the expression of four genes is analyzed to calculate the classifier value). In some embodiments, the four-gene classifier has a greater than about 75%, 90%, 95%, 97%, or 99% confidence interval for distinguishing non-melanoma from melanoma. In some embodiments, the four-gene classifier has a greater than about 75%, 90%, 95%, 97%, or 99% confidence interval for distinguishing melanoma in situ from invasive melanoma. In some embodiments, the four-gene classifier has a greater than about 75%, 90%, 95%, 97%, or 99% confidence interval for distinguishing non-melanoma, melanoma in situ, and invasive melanoma.

In some embodiments, a gene classifier for the diagnosis of melanoma comprising five or more genes is analyzed. In some embodiments, the gene classifier for the diagnosis of melanoma comprising five or more genes has a greater than about 75%, 90%, 95%, 97%, or 99% confidence interval for distinguishing non-melanoma from melanoma. In some embodiments, the gene classifier for the diagnosis of melanoma comprising five or more genes has a greater than about 75%, 90%, 95%, 97%, or 99% confidence interval for distinguishing melanoma in situ from invasive melanoma. In some embodiments, the gene classifier for the diagnosis of melanoma comprising five or more genes has a greater than about 75%, 90%, 95%, 97%, or 99% confidence interval for distinguishing non-melanoma, melanoma in situ, and invasive melanoma. In some embodiments, the one or more genes analyzed according to the methods described herein have an area under the curve (AUC) of a receiver operating characteristic (ROC) curve (AUC ROC) of greater than about 0.5. In some embodiments, the one or more genes analyzed according to the methods described herein have an AUC ROC of greater than about 0.5, greater than about 0.6, greater than about 0.7, greater than about 0.8, or greater than about 0.9. In some embodiments, the one or more genes analyzed have an AUC ROC of greater than about 0.95.

In some embodiments, a gene classifier analyzed according to the methods described herein has an area under the curve (AUC) of a receiver operating characteristic (ROC) curve (AUC ROC) of greater than about 0.5. In some embodiments, a gene classifier analyzed according to the methods and gene classifiers described herein has an AUC ROC of greater than about 0.5, greater than about 0.6, greater than about 0.7, greater than about 0.8, or greater than about 0.9. In some embodiments, a gene classifier analyzed according to the methods described herein has an AUC ROC of greater than about 0.95.

In some embodiments, a two-gene classifier for the diagnosis of melanoma analyzed according to the methods described herein has an area under the curve (AUC) of a receiver operating characteristic (ROC) curve (AUC ROC) of greater than about 0.5. In some embodiments, a two-gene classifier for the diagnosis of melanoma analyzed according to the methods described herein has an AUC ROC of greater than about 0.5, greater than about 0.6, greater than about 0.7, greater than about 0.8, or greater than about 0.9. In some embodiments, a two-gene classifier for the diagnosis of melanoma analyzed according to the methods described herein has an AUC ROC of greater than about 0.95. In some embodiments, the two-gene classifier is LINC00518 and CMIP. In some embodiments, the two-gene classifier is LINC00518 and TMEM80. In some embodiments, the two-gene classifier is LINC00518 and ACTN4. In some embodiments, the two-gene classifier is LINC00518 and NAMPT. In some embodiments, the two-gene classifier is LINC00518 and TTC3. In some embodiments, the two-gene classifier is LINC00518 and HLA-C. In some embodiments, the two-gene classifier is LINC00518 and PRAME. In some embodiments, the two-gene classifier is LINC00518 and TRIB2. In some embodiments, the two-gene classifier is LINC00518 and GPM6B. In some embodiments, the two-gene classifier is TRIB2 and NAMPT. In some embodiments, the two-gene classifier is TRIB2 and KIT. In some embodiments, the two-gene classifier is TRIB2 and ACTN4. In some embodiments, a two-gene classifier that is LINC00518 and CMIP analyzed according to the methods described herein has an AUC ROC of greater than 0.95.

In some embodiments, a three-gene classifier analyzed according to the methods described herein has an area under the curve (AUC) of a receiver operating characteristic (ROC) curve (AUC ROC) of greater than about 0.5. In some embodiments, a three-gene classifier analyzed according to the methods described herein has an AUC ROC of greater than about 0.5, greater than about 0.6, greater than about 0.7, greater than about 0.8, or greater than about 0.9. In some embodiments, a three-gene classifier analyzed according to the methods described herein has an AUC ROC of greater than about 0.95. In some embodiments, the three-gene classifier is ACTN4, LINC00518 and PRAME. In some embodiments, the three-gene classifier that is ACTN4, LINC00518 and PRAME analyzed according to the methods described herein has an AUC ROC of greater than about 0.95.

In some embodiments, the one or more genes analyzed according to the methods described herein have a sensitivity of greater than about 50%. In some embodiments, the one or more genes analyzed according to the methods described herein have a sensitivity greater than about 60%, greater than about 65%, greater than about 70%, greater than about 75%, greater than about 80%, greater than about 85%, greater than about 90%, greater than about 93%, greater than about 95%, greater than about 96%, greater than about 97%, greater than about 98%, or greater than about 99%. In some embodiments, the one or more genes analyzed according to the methods described herein have a sensitivity of greater than about 95%.

In some embodiments, a gene classifier analyzed according to the methods described herein has a sensitivity of greater than about 50%. In some embodiments, a gene classifier analyzed according to the methods described herein has a sensitivity of greater than about 60%, greater than about 70%, greater than about 80%, greater than about 90%, greater than about 93%, greater than about 95%, greater than about 96%, greater than about 97%, greater than about 98%, or greater than about 99%. In some embodiments, a gene classifier analyzed according to the methods described herein has a sensitivity of greater than about 95%.

In some embodiments, a two-gene classifier analyzed according to the methods described herein has a sensitivity of greater than about 50%. In some embodiments, a two-gene classifier analyzed according to the methods described herein has a sensitivity of greater than about 60%, greater than about 65%, greater than about 70%, greater than about 75%, greater than about 80%, greater than about 85%, greater than about 90%, greater than about 93%, greater than about 95%, greater than about 96%, greater than about 97%, greater than about 98%, or greater than about 99%. In some embodiments, a two-gene classifier analyzed according to the methods described herein has a sensitivity of greater than about 95%. In some embodiments, the two-gene classifier is LINC00518 and CMIP. In some embodiments, the two-gene classifier is LINC00518 and TMEM80. In some embodiments, the two-gene classifier is LINC00518 and ACTN4. In some embodiments, the two-gene classifier is LINC00518 and NAMPT. In some embodiments, the two-gene classifier is LINC00518 and TTC3. In some embodiments, the two-gene classifier is LINC00518 and HLA-C. In some embodiments, the two-gene classifier is LINC00518 and PRAME. In some embodiments, the two-gene classifier is LINC00518 and TRIB2. In some embodiments, the two-gene classifier is LINC00518 and GPM6B. In some embodiments, the two-gene classifier is TRIB2 and NAMPT. In some embodiments, the two-gene classifier is TRIB2 and KIT. In some embodiments, the two-gene classifier is TRIB2 and ACTN4. In some embodiments, a two-gene classifier that is LINC00518 and CMIP analyzed according to the methods described herein has a sensitivity of greater than 95%. In some embodiments, a three-gene classifier analyzed according to the methods described herein has a sensitivity of greater than about 50%. In some embodiments, a three-gene classifier analyzed according to the methods described herein has a sensitivity of greater than about 60%, greater than about 70%, greater than about 80%, greater than about 90%, greater than about 93%, greater than about 95%, greater than about 96%, greater than about 97%, greater than about 98%, or greater than about 99%. In some embodiments, a three-gene classifier analyzed according to the methods described herein has a sensitivity of greater than about 95%. In some embodiments, a three-gene classifier that is ACTN4, LINC00518 and PRAME analyzed according to the methods described herein has a sensitivity of greater than about 95%.

In some embodiments, the one or more genes analyzed according to the methods described herein have a specificity of greater than about 50%. In some embodiments, the one or more genes analyzed according to the methods described herein have a specificity of greater than about 60%, greater than about 65%, greater than about 70%, greater than about 75%, greater than about 80%, greater than about 85%, greater than about 90%, greater than about 93%, greater than about 95%, greater than about 96%, greater than about 97%, greater than about 98%, or greater than about 99%. In some embodiments, the one or more genes analyzed according to the methods described herein have a specificity of greater than 95%.

In some embodiments, a gene classifier analyzed according to the methods described herein has a specificity of greater than about 50%. In some embodiments, a gene classifier analyzed according to the methods described herein has a specificity of greater than about 60%, greater than about 70%, greater than about 80%, greater than about 90%, greater than about 93%, greater than about 95%, greater than about 96%, greater than about 97%, greater than about 98%, or greater than about 99%. In some embodiments, a gene classifier analyzed according to the methods described herein has a specificity of greater than about 95%.

In some embodiments, a two-gene classifier analyzed according to the methods described herein has a specificity of greater than about 50%. In some embodiments, a two-gene classifier analyzed according to the methods described herein has a specificity of greater than about 60%, greater than about 65%, greater than about 70%, greater than about 75%, greater than about 80%, greater than about 85%, greater than about 90%, greater than about 93%, greater than about 95%, greater than about 96%, greater than about 97%, greater than about 98%, or greater than about 99%. In some embodiments, a two-gene classifier analyzed according to the methods described herein has a specificity of greater than about 95%. In some embodiments, the two-gene classifier is LINC00518 and CMIP. In some embodiments, the two-gene classifier is LINC00518 and TMEM80. In some embodiments, the two-gene classifier is LINC00518 and ACTN4. In some embodiments, the two-gene classifier is LINC00518 and NAMPT. In some embodiments, the two-gene classifier is LINC00518 and TTC3. In some embodiments, the two-gene classifier is LINC00518 and HLA-C. In some embodiments, the two-gene classifier is LINC00518 and PRAME. In some embodiments, the two-gene classifier is LINC00518 and TRIB2. In some embodiments, the two-gene classifier is LINC00518 and GPM6B. In some embodiments, the two-gene classifier is TRIB2 and NAMPT. In some embodiments, the two-gene classifier is TRIB2 and KIT. In some embodiments, the two-gene classifier is TRIB2 and ACTN4. In some embodiments, a two-gene classifier that is LINC00518 and CMIP analyzed according to the methods described herein has a specificity of greater than 95%.

In some embodiments, a three-gene classifier analyzed according to the methods described herein has a specificity of greater than about 50%. In some embodiments, a three-gene classifier analyzed according to the methods described herein has a specificity of greater than about 60%, greater than about 70%, greater than about 80%, greater than about 90%, greater than about 93%, greater than about 95%, greater than about 96%, greater than about 97%, greater than about 98%, or greater than about 99%. In some embodiments, a three-gene classifier analyzed according to the methods described herein has a specificity of greater than about 95%. In some embodiments, a three-gene classifier that is ACTN4, LINC00518 and PRAME analyzed according to the methods described herein has a specificity of greater than about 95%.

In some embodiments, a gene classifier analyzed according to the methods described herein has a sensitivity of greater than about 95% and a specificity of greater than about 70%. In some embodiments, a gene classifier analyzed according to the methods described herein has a sensitivity of greater than about 95% and a specificity of greater than about 80%. In some embodiments, a gene classifier analyzed according to the methods described herein has a sensitivity of greater than about 99% and a specificity of greater than about 80%.

In some embodiments, a two-gene classifier analyzed according to the methods described herein has a sensitivity of greater than about 95% and a specificity of greater than about 70%. In some embodiments, a two-gene classifier analyzed according to the methods described herein has a sensitivity of greater than about 95% and a specificity of greater than about 80%. In some embodiments, a two-gene classifier analyzed according to the methods described herein has a sensitivity of greater than about 99% and a specificity of greater than about 80%.

In some embodiments, a two-gene classifier that is LINC00518 and CMIP analyzed according to the methods described herein has a sensitivity of greater than about 95% and a specificity of greater than about 70%. In some embodiments, a two-gene classifier that is LINC00518 and CMIP analyzed according to the methods described herein has a sensitivity of greater than about 95% and a specificity of greater than about 80%. In some embodiments, a two-gene classifier that is LINC00518 and CMIP analyzed according to the methods described herein has a sensitivity of greater than about 99% and a specificity of greater than about 80%.

In some embodiments, a limited amount of total nucleic acid is required for performing the methods described herein. In some embodiments, the total nucleic acid required for performing the methods described herein is less than about 1000 ng. In some embodiments, the total nucleic acid required for performing the methods described herein is less than about 500 ng. In some embodiments, the total nucleic acid required for performing the methods described herein is less than about 100 ng. In some embodiments, the total nucleic acid required for performing the methods described herein is less than about 1 ng. In some embodiments, the total nucleic acid required for performing the methods described herein is less than about 500 pg. In some embodiments, the total nucleic acid required for performing the methods described herein is less than about 250 pg. In some embodiments, the nucleic acid is RNA.

In some embodiments, a gene classifier analyzed according to the methods described herein has a sensitivity of greater than about 95% and a specificity of greater than about 70% when analyzed with about 250-1000 pg input RNA. In some embodiments, a gene classifier analyzed according to the methods described herein has a sensitivity of greater than 95% and a specificity of greater than 80% when analyzed with about 250-1000 pg input RNA. In some embodiments, a gene classifier analyzed according to the methods described herein has a sensitivity of greater than 99% and a specificity of greater than about 80% when analyzed with about 250-1000 pg input RNA.

In some embodiments, a two-gene classifier analyzed according to the methods described herein has a sensitivity of greater than about 95% and a specificity of greater than 70% when analyzed with about 250-1000 pg input RNA. In some embodiments, a two-gene classifier analyzed according to the methods described herein has a sensitivity of greater than about 95% and a specificity of greater than about 80% when analyzed with about 250-1000 pg input RNA. In some embodiments, a two-gene classifier analyzed according to the methods described herein has a sensitivity of greater than about 99% and a specificity of greater than about 80% when analyzed with about 250-1000 pg input RNA.

In some embodiments, a two-gene classifier that is LINC00518 and CMIP analyzed according to the methods described herein has a sensitivity of greater than about 95% and a specificity of greater than about 70% when analyzed with about 250-1000 pg input RNA. In some embodiments, a two-gene classifier that is LINC00518 and CMIP analyzed according to the methods described herein has a sensitivity of greater than about 95% and a specificity of greater than about 80% when analyzed with about 250-1000 pg input RNA. In some embodiments, a two-gene classifier that is LINC00518 and CMIP analyzed according to the methods described herein has a sensitivity of greater than about 99% and a specificity of greater than about 80% when analyzed with about 250-1000 pg input RNA.

Nucleic acid molecules may be analyzed in any number of ways known in the art. For example, the presence of nucleic acid molecules can be detected by DNA-DNA or DNA-RNA hybridization or amplification using probes or fragments of the specific nucleic acid molecule. Nucleic acid amplification based assays involve the use of oligonucleotides or oligomers based on the nucleic acid sequences to detect transformants containing the specific DNA or RNA.

In some embodiments, the methods further comprise detecting a relative amount of the gene product compared to a control. In some embodiments, the gene product is a nucleic acid molecule or a protein. In some embodiments, the nucleic acid molecule is an RNA molecule. In some embodiments, the control is the relative amount of the gene product expressed in a normal epidermal skin sample. In some embodiments, the relative amount of the gene product is decreased compared to the control by about 2-fold, 3-fold, 4-fold, 5-fold, 10-fold, 20-fold, 30-fold, 40-fold, 50-fold, 60-fold, 70-fold, 80-fold, 90-fold, or 100-fold. In some embodiments, the relative amount of the gene product is increased compared to the control by about 2-fold, 3-fold, 4-fold, 5-fold, 10-fold, 20-fold, 30-fold, 40-fold, 50-fold, 60-fold, 70-fold, 80-fold, 90-fold, or 100-fold.

In one embodiment, analysis of the nucleic acid molecules includes genetic analysis to determine the nucleotide sequence of a gene. Since a difference in length or sequence between DNA fragments isolated from a sample and those of known sequences are due to an insertion, deletion, or substitution of one or more nucleotides, the determination of nucleic acid sequences provides information concerning mutations which have absolute influence on the physiology of the disease state in the subject. These mutations may also include a transposition or an inversion and are difficult to detect by other techniques than direct sequencing. Accordingly, the methods of the present invention may be used to detect genetic mutations in one or more genes comprising: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, or VIM, for diagnosis and/or characterization of a skin lesion in a subject.

A variety of protocols for detecting and measuring the expression of nucleic acid molecules, using either polyclonal or monoclonal antibodies specific for the protein expression product are known in the art. Examples include enzyme-linked immunosorbent assay (ELISA), radioimmunoassay (RIA), and fluorescence activated cell sorting (FACS). These and other assays are described, among other places, in Hampton, R. et al. (1990; Serological Methods, a Laboratory Manual, APS Press, St Paul, Minn.) and Maddox, D. E. et al. (1983; J. Exp. Med. 158:1211-1216).

In order to provide a basis for the diagnosis or characterization of disease associated with expression of the nucleic acid molecules of the invention, a normal or standard profile for expression is established. Standard hybridization may be quantified by comparing the values obtained from subjects of known skin characterization (e.g., from subjects either having melanoma, having dysplastic nevi, and/or having solar lentigines). Standard values obtained from such samples may be compared with values obtained from samples from subjects having skin lesions that are suspected of being melanoma. Deviation between standard and subject values is used to establish the presence of disease.

Accordingly, in one aspect of the invention, a non-invasive sampling method is provided for the characterization of skin lesion on the skin. In one embodiment, a sample set of pigmented skin lesions is created. Each sample consists of nucleic acid molecules recovered by tape stripping or biopsy sample of the superficial epidermis overlying the lesion. In addition to tape striping, a standard biopsy of the same lesion may also be performed, along with accompanying histology and diagnosis. Nucleic acid molecules recovered by tape stripping the superficial epidermis of normal skin will serve as a negative control.

In another aspect, the invention provides a method of distinguishing melanoma from solar lentigo and/or dysplastic nevi and/or normal pigmented skin in a subject. In one embodiment, the method includes analyzing a nucleic acid molecule from one or more genes selected from ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, VIM, or any combination thereof. A target area of the skin of a subject that suspected of being melanoma is assayed for expression of a large number of genes. Analyzing expression includes any qualitative or quantitative method for detecting expression of a gene, many of which are known in the art. The method can include analyzing expression of specific markers by measuring expression of the markers using a quantitative method, or by using a qualitative method. Non-limiting methods for analyzing polynucleotides and polypeptides are discussed below.

In another aspect, the invention provides a method of distinguishing solar lentigines from dysplastic nevi and/or basal cell carcinoma and/or normal pigmented skin in a subject. In one embodiment, the method includes analyzing a nucleic acid molecule from one or more genes selected from ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, or VIM, or any combination thereof.

In a related aspect, the invention provides a method for diagnosing various disease states in a subject by identifying new diagnostic markers, specifically the classification and diagnosis of melanoma. In addition, the invention provides a method for distinguishing solar lentigines from dysplastic nevi and/or lentigo maligna and/or normal skin. Thus, the invention provides a method for diagnosing various disease states in a subject by identifying new diagnostic markers, specifically the classification and diagnosis of melanoma. By identifying gene sets that are unique to a given state, these differences in the genetic expression can be utilized for diagnostic purposes. In one embodiment, the nucleic acid molecule is RNA, including messenger RNA (mRNA) that is isolated from a sample from the subject. Up-regulated and down-regulated gene sets for a given disease state may be subsequently combined. The combination enables those of skill in the art to identify gene sets or panels that are unique to a given disease state. Such gene sets are of immense diagnostic value as they can be routinely used in assays that are simpler than microarray analysis (for example “real-time” quantitative PCR). Such gene sets also provide insights into pathogenesis and targets for the design of new drugs.

A reference database containing a number of reference projected profiles is also created from skin samples of subjects with known states, such as normal (i.e., non-melanoma) and various skin cancer disease states and/or pigmented non-cancer states. The projected profile is then compared with the reference database containing the reference projected profiles. If the projected profile of the subject matches best with the profile of a particular disease state in the database, the subject is diagnosed as having such disease state. Various computer systems and software can be utilized for implementing the analytical methods of this invention and are apparent to one of skill in the art. Exemplary software programs include, but are not limited to, Cluster & TreeView (Stanford, URLs: rana.lbl.gov or microarray.org), GeneCluster (MIT/Whitehead Institute, URL: MPR/GeneCluster/GeneCluster.html), Array Explorer (SpotFire Inc, URL: spotfire.com/products/scicomp.asp#SAE) and GeneSpring (Silicon Genetics Inc, URL: sigenetics.com/Products/GeneSpring/index.html) (for computer systems and software, see also U.S. Pat. No. 6,203,987, incorporated herein by reference).

In another aspect, the methods of the present invention involve in situ analysis of the skin lesion for characterization thereof. For in situ methods, nucleic acid molecules do not need to be isolated from the subject prior to analysis. In one embodiment, detectably labeled probes are contacted with a cell or tissue of a subject for visual detection of expressed RNA to characterize the skin lesion.

### Applications of the Methods

Methods provided herein which isolate and detect a nucleic acid sample from an epidermal sample of a skin lesion have utility not only in characterizing the skin lesion, but also in diagnosing, and prognosing melanoma as well as monitoring response of a subject to treatment. In some embodiments, these methods are used to identify a predictive skin marker to identify a lesion as being melanoma and/or a patient, that will respond to treatment for melanoma.

For example, by comparing the projected profile prior to treatment with the profile after treatment. In one embodiment, the method characterizes a cancer as melanoma metastasis based on analysis of one or more nucleic acid molecules selected from ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, or VIM. In another embodiment, the method characterizes a solar lentigo based on analysis of one or more nucleic acid molecules selected from ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, or VIM. It is known that in many cases, by the time a diagnosis of melanoma is established in a subject, metastasis has already occurred since melanomas contain multiple cell populations characterized by diverse growth rates, karyotypes, cell-surface properties, antigenicity, immunogenicity, invasion, metastasis, and sensitivity to cytotoxic drugs or biologic agents. Thus, the present invention may be used to characterize cancer of an organ as having metastasized from melanoma.

In a related aspect, the methods of the present invention can also be useful for determining an appropriate treatment regimen for a subject having a specific cancer or melanoma. In another related aspect, the methods of the present invention can also be useful for determining an appropriate treatment regimen for a subject having solar lentigo. Thus, the methods of the invention are useful for providing a means for practicing personalized medicine, wherein treatment is tailored to a subject based on the particular characteristics of the cancer or skin lesion in the subject. The method can be practiced, for example, by first determining whether the skin lesion is melanoma or solar lentigo, as described above.

The sample of cells examined according to the present method can be obtained from the subject to be treated, or can be cells of an established cancer cell line of the same type as that of the subject. In one aspect, the established cell line can be one of a panel of such cell lines, wherein the panel can include different cell lines of the same type of disease and/or different cell lines of different diseases associated with expression of the genes of interest. Such a panel of cell lines can be useful, for example, to practice the present method when only a small number of cells can be obtained from the subject to be treated, thus providing a surrogate sample of the subject's cells, and also can be useful to include as control samples in practicing the present methods.

Once disease and/or skin lesion characterization is established and a treatment protocol is initiated, the methods of the invention may be repeated on a regular basis to monitor the expression profiles of the genes of interest in the subject. The results obtained from successive assays may be used to show the efficacy of treatment over a period ranging from several days to months. Accordingly, another aspect of the invention is directed to methods for monitoring a therapeutic regimen for treating a subject having skin cancer. A comparison of the expression profile or mutations in the nucleic acid sequence of the nucleic acid molecule prior to and during therapy will be indicative of the efficacy of the therapy. Therefore, one skilled in the art will be able to recognize and adjust the therapeutic approach as needed.

Treatment regimens for melanoma include chemotherapeutic agents, radiation, anti-angiogenic compounds, or other agents for treating cancer in combination with immunization strategies. Other suitable treatments for melanoma include, for example, surgery, adjuvant radiation therapy, adjuvant interferon alfa-2b, corticosteroids, systemic therapy, dacarbazine, combination chemotherapies, such as cisplatin, carmustine, dacarbazine, and tamoxifen. In some embodiments, the melanoma is treated with Yervoy, nivolumab, MPDL3280, or a combination thereof. Other suitable treatments are known in the art.

The efficacy of a therapeutic regimen for treating a cancer over time can be identified by an absence of symptoms or clinical signs of the particular cancer in a subject at the time of onset of therapy. In subjects diagnosed as having the particular cancer, the efficacy of a method of the invention can be evaluated by measuring a lessening in the severity of the signs or symptoms in the subject or by the occurrence of a surrogate end-point for the disorder.

In addition, such methods may help identify an individual as having a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

In certain embodiments, expression of a gene product in the epidermal sample is predictive of response to treatment if expression of the gene product at the first time point is different in subjects that respond to treatment compared to subjects that do not respond to treatment. It will be understood that a variety of statistical analysis can be performed to identify a statistically significant association between expression of the gene product and response of the subject to the treatment. In some embodiment, the expression of the gene product, in certain examples, is elevated in subjects that will not respond to treatment. Furthermore, expression of the gene product can predict a level of response to treatment, for example partial or temporary response to treatment versus a full response. In some embodiments, a target gene product is a nucleic acid molecule. In some embodiments, a target gene product is a polypeptide.

In some embodiments, provided herein is a non-invasive method for predicting response to treatment for melanoma, including applying an adhesive tape to the skin of a subject afflicted with melanoma in a manner sufficient to isolate an epidermal sample that includes a gene product. In some embodiments, a target gene product is detected in the epidermal sample, whose expression is indicative of a response to treatment, thereby predicting response to treatment for melanoma. In some embodiments, a target gene product is a nucleic acid molecule. In some embodiments, a target gene product is a polypeptide.

Certain embodiments provided herein, are based in part on the discovery that the expression of certain genes can be used to monitor response to therapy. Accordingly, in another embodiment, provided herein is a method for monitoring a response of a human subject to treatment for melanoma, including applying an adhesive tape to the skin of the subject being treated for the disease or condition at a first time point and at least a second time point, in a manner sufficient to isolate an epidermal sample adhering to the adhesive tape at the first time point and at the second time point. In some embodiments, the epidermal sample includes a gene product, wherein a change in expression of the gene product between the first time point and the second time point is indicative of a change in severity or level of melanoma. In some embodiments, a target gene product is a nucleic acid molecule. In some embodiments, a target gene product is a polypeptide.

In some embodiments, provided herein is a method for detecting a response of a subject to treatment for melanoma or monitoring the response of a subject to treatment for melanoma over a period of time, comprising: treating the subject for a skin disease or condition state; applying an adhesive tape to the skin of the subject in a manner sufficient to isolate an epidermal sample, wherein the epidermal sample includes a gene product; and detecting a target gene product in the sample. Expression of the target gene product is informative regarding pathogenesis of melanoma. Therefore, the method identifies a response of the subject to treatment for melanoma. In some embodiments, a target gene product is a nucleic acid molecule. In some embodiments, a target gene product is a polypeptide.

In some embodiments, the detection of the gene product is a qualitative detection of whether the target gene product is expressed. In some embodiments, the detection of the target gene product is quantitative assessment of the expression level of the target gene product. In some embodiments, the method is performed both prior to treatment and after treatment. In some embodiments, the method is performed after treatment, but before a change in severity or level of melanoma is observed. In some embodiments, the method is performed at multiple time point during treatment.

Time points for the monitoring and response-to-treatment methods provided herein, include any interval of time. In some embodiments, the time points are 1 day, 2 days, 3 days, 4 days, 5 days 6 days, 1 week, 2 weeks, 3, weeks, 4 weeks, 1 month, 2 months, 3 months, 4 months, 5 months, 6 months, 7 months, 8 months, 9 months, 10 months, 11 months, 1 year, 2 years or longer apart.

In some embodiments, skin samples are obtained at any number of time points, including 2, 3, 4, 5, 6, 7, 8, 9, 10, 11, 12, or more time points.

In some embodiments, comparison of expression analysis data from different time points is performed using any of the known statistical methods for comparing data points to assess differences in the data, including time-based statistical methods such as control charting. In some embodiments, the identity, severity or level of melanoma is identified in the time series, for example, by comparing expression levels to a cut-off value, or by comparing changes in expression levels to determine whether they exceed a cut-off change value, such as a percent change cut-off value. In certain aspects, the first time point is prior to treatment, for example, prior to administration of a therapeutic agent, and the second time point is after treatment.

In some embodiments, the change in expression levels of at least one gene product is an increase or decrease in expression. Depending on the target gene product, an increase or decrease indicates a response to treatment, or a lack of response. For example, in some embodiments, the gene product is a nucleic acid from one or more genes selected from ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, and VIM, and a decrease in expression at the second time point as compared to the first time point is indicative of positive response to treatment for melanoma. As another example, in some embodiments, the gene product detected is a polypeptide that is expressed by a gene selected from ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, and VIM, and a decrease in expression at the second time point as compared to the first time point is indicative of positive response to treatment for melanoma. In some embodiments, the gene product is a nucleic acid that encodes a protein such as a protein expressed by a gene selected from ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, and VIM, and an increase in expression at the second time point as compared to the first time point is indicative of positive response to treatment for melanoma. As another example, in some embodiments, the gene product detected is a polypeptide that is expressed by a gene selected from ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, and VIM, and an increase in expression at the second time point as compared to the first time point is indicative of positive response to treatment for melanoma.

In some embodiments, more than one target gene product is detected. In some embodiments, a population of target gene products are detected. In some embodiments, the method for detecting a population of target gene products is performed using a microarray.

In some embodiments where expression of more than 1 gene is analyzed, the detection is performed using a microarray. In some examples, the microarray includes an array of sequence specific nucleic acid probes. In some embodiments, the microarray includes an array of sequence specific nucleic acid probes directed to 2 or more, 3 or more, 4 or more, 5 or more, 6 or more, 7 or more, 8 or more, 9 or more, 10 or more, 15 or more, 20 or more, 25 or more, 30 or more, 35 or more, 40 or more, 45 or more or all of the genes selected from ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, and VIM.

In some embodiments, the relative amount of the gene product is increased in an epidermal skin sample from a melanoma lesion or an epidermal skin sample from a suspected melanoma lesion compared to a control by about 2-fold, 3-fold, 4-fold, 5-fold, 10-fold, 20-fold, 30-fold, 40-fold, 50-fold, 60-fold, 70-fold, 80-fold, 90-fold, or 100-fold. In some embodiments, the relative amount of the gene product is decreased in an epidermal skin sample from a melanoma lesion or an epidermal skin sample from a suspected melanoma lesion compared to a control by about 2-fold, 3-fold, 4-fold, 5-fold, 10-fold, 20-fold, 30-fold, 40-fold, 50-fold, 60-fold, 70-fold, 80-fold, 90-fold, or 100-fold. In some embodiments, the control is a normal skin sample. In some embodiments, the control is a value obtained from a database of relative expression values. In some embodiments, the control is a value obtained from a known relative expression values.

In some embodiments, expression of a target gene believed to be involved in melanoma is detected in a skin lesion using a tape stripping method provided herein. In some embodiments, if expression or elevated expression is detected, a treatment is administered to the subject that blocks a function of the target gene. Accordingly, in some embodiments, the methods provided herein are used to determine whether the subject is likely to respond to treatment with a biologic that targets a particular gene that exhibits elevated expression in a skin lesion.

### Kits

In another embodiment, provided herein are kits that include one or more reagents or devices for the performance of the methods disclosed herein. In some embodiments, provided is a kit for isolation and detection of a nucleic acid from an epidermal sample, such as a pigmented skin lesion.

In some embodiments, the kit includes an adhesive tape for performing methods provided herein. In some embodiments, the kit includes an adhesive tape for tape stripping skin, such as rubber-based, pliable adhesive tape. Accordingly, in some embodiments, provided herein is a kit, including a pliable adhesive tape made up at least in part, of a non-polar polymer. In certain aspects, the tape includes a rubber adhesive. In an illustrative example, the tape can be skin harvesting tape available (Product No. 90068) from Adhesives Research, Inc (Glen Rock, Pa.). In some embodiments, the kit includes instructions for performing tape strippings or for analyzing gene expression.

In some embodiments, the kit includes nucleic acid or polypeptide isolation reagents.

In some embodiments, the kit includes one or more detection reagents, for example probes and/or primers for amplification of, or hybridization to, a target nucleic acid sequence whose expression is related to melanoma. In some embodiments, the kit includes primers and probes for control genes, such as housekeeping genes. In some embodiments, the primers and probes for control genes are used, for example, in ΔCₜ calculations. In some embodiments, the probes or primers are labeled with an enzymatic, florescent, or radionuclide label. In some embodiments, the probe binds to a target nucleic acid molecule encoding a protein. In some embodiments, the probe is an antibody or ligand that binds the encoded protein. In some embodiments, probes are spotted on a microarray. In some embodiments, the microarray is provided in the kit.

The term “detectably labeled deoxyribonucleotide” refers to a deoxyribonucleotide that is associated with a detectable label for detecting the deoxyribonucleotide. For example, the detectable label may be a radiolabeled nucleotide or a small molecule covalently bound to the nucleotide where the small molecule is recognized by a well-characterized large molecule. Examples of these small molecules are biotin, which is bound by avidin, and thyroxin, which is bound by anti-thyroxin antibody. Other labels are known to those of ordinary skill in the art, including enzymatic, fluorescent compounds, chemiluminescent compounds, phosphorescent compounds, and bioluminescent compounds.

In some embodiments, the kit includes one or more primer pairs, including a forward primer that selectively binds upstream of a gene whose expression is associated with psoriasis or irritant dermatitis, for example, on one strand, and a reverse primer, that selectively binds upstream of a gene involved in psoriasis or irritant dermatitis on a complementary strand. Primer pairs according to this aspect of the invention are typically useful for amplifying a polynucleotide that corresponds to a skin marker gene associated with melanoma using amplification methods described herein.

In some embodiments, a kit provided herein includes a carrier means being compartmentalized to receive in close confinement one or more containers such as vials, tubes, and the like, each of the containers comprising one of the separate elements to be used in a method provided herein. In some embodiments, a second container includes, for example, a lysis buffer. In some embodiments, the kit includes a computer-type chip on which the lysis of the cell will be achieved by means of an electric current.

In another embodiment, a kit of the invention includes a probe that binds to a portion of a nucleic acid molecule of a gene selected from ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, or VIM. In another embodiment, the kit further includes a microarray that contains at least a fragment of a gene or a nucleic acid molecule or a protein product of any one of the genes selected from ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, or VIM. In some embodiments, many reagents may be provided in a kit of the invention, only some of which should be used together in a particular reaction or procedure. For example, multiple primers may be provided, only two of which are needed for a particular application.

The following examples are provided to further illustrate the advantages and features of the present invention, but are not intended to limit the scope of the invention. While they are typical of those that might be used, other procedures, methodologies, or techniques known to those skilled in the art may alternatively be used.

### Systems

Disclosed herein, in some embodiments, is a system for characterizing a skin lesion in a subject, comprising analyzing a nucleic acid molecule from one or more genes selected from: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, and VIM, in a sample of the skin lesion, thereby producing a characterization of the skin lesion. In some embodiments, two or more genes selected from: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, and VIM are analyzed, In some embodiments, the genes are selected from LINC00518, TRIB2, KIT, SDCBP, TYR, NAMPT, ACTN4, EDNRB, GPM6B, CNN2, MCOLN3, PRAME, TMEM80, TTC3, and CMIP, and combinations thereof.

In some embodiments, disclosed herein is a system for characterizing a skin lesion in a subject in a subject, comprising: (a) a computer processing device, optionally connected to a computer network; and (b) a software module executed by the computer processing device to analyzing a nucleic acid molecule from one or more genes selected from: ACTB, ACTN4, B2M, LINC00518, CNN2, CMIP, EDNRB, GPM6B, KIT, MCOLN3, NAMPT, PPIA, SDCBP, TTC3, TMEM80, TRIB2, TYR, ANKRD11, CASC3, CCL18, CMPK2, CTBP2, DCT, EZR, FOS, FTL, HLA-A, HLA-C, HOXA9, IFI6, IRF9, ISG15, KRT17, KTN1, MAFB, MAFK, MAL, MALAT1, MAP1B, MARCKS, MLANA, RNA00188, OTUB1, OVOS2, PKD1P1, PMEL, PRAME, PTPN14, SAT1, SDHA, SIRPA, SOX10, TFRC, TYRP1, UBE2B, in a sample from a skin lesion, to a standard or control.

### EXAMPLES

### Example 1

### Tape Stripping to Recover Nucleic Acids from Pigmented Lesions

The following procedure was used to recover nucleic acids from pigmented lesions and/or skin suspected of melanoma of a subject. In contrast to normal skin, lesional skin should have a preoperative biopsy diameter of greater than or equal to about 6 mm, but less than that of the tape disc. Multiple lesions must be at least about 4 mm apart. The area of tape that touches the lesion should be generously demarcated on the tape with an insoluble ink pen so that this area may be cut away from the surrounding tape at the laboratory as part of the RNA extraction procedure.

As above, tapes were handled with gloved hands at all times. The site is shaved, if necessary, to remove non-vellus hairs. The site is then cleansed with an alcohol wipe (70% isopropyl alcohol). The site is then air-dried completely before application of the tape. It is recommended to approximately 2 minutes to ensure the site is completely dry before application of the tape.

Apply the tape to the skin site. If more than one tape is used, apply tapes in sequential order starting from the left side. Use a surgical skin marker and/or a water soluble marker to mark the location of the tape on the skin in order to align subsequent tapes. Apply the tape to the suspect lesion, which should have a diameter that is greater than or equal to about 6 mm.

Start the tape harvesting procedure by applying pressure directly over the lesion and avoiding surrounding normal skin (press on the tape firmly). Ensure that the skin is held taut to ensure that the tape does not move while applying pressure. Using a marking pen, demarcate a zone around the lesion such that the area of the lesion is encompassed within the inked boundary and the boundary is approximately 1 mm from the lesion border.

Remove the tape slowly in one direction. Place the edge of the tape onto the adhesive strip with cells facing down to protect the sample. Put a second tape on the same site following directions provided above. Repeat until the lesion has been stripped a total of at least four times, unless otherwise specified in the protocol. Place the strip into a storage bag and immediately place the samples on dry ice or into storage at −20° C. or below until analysis.

### Example 2

### RNA Quantitation and Profiling

The study was divided into two separate phases, a sample collection and characterization phase (phase 1) and an RNA profiling phase (phase 2). In phase 1 the tape stripped specimens and biopsied sample collections were performed by the principal investigator or trained individuals delegated by the principal investigator to obtain the biopsy sample at various sites. All biopsies are subject to standard histopathologic analysis. The RNA profiling phase (Phase 2), includes, but is not limited to RNA purification and hybridization to DNA microarrays for gene expression profiling.

Materials and reagents. Adhesive tape was purchased from Adhesives Research (Glen Rock, Pa.) in bulk rolls. These rolls were custom fabricated into small circular discs, 17 millimeters in diameter, by Diagnostic Laminations Engineering (Oceanside, Calif.). Human spleen total RNA was purchased from Ambion (catalogue #7970; Austin, Tex.). RNeasy RNA extraction kit was purchased from Qiagen (Valencia, Calif.). Reverse transcriptase, PCR primers and probes, and TaqMan Universal Master Mix, which included all buffers and enzymes necessary for the amplification and fluorescent detection of specific cDNAs, were purchased from Applied Biosystems (Foster City, Calif.). MELT total nucleic acid isolation system was purchased from Ambion (Austin, Tex.).

RNA isolation. RNA was extracted from tapes using either pressure cycling technology (PCT; Garrett, Tao et al. 2002; Schumacher, Manak et al. 2002) or MELT total nucleic acid system. Tapes were extracted in pairs by insertion into a PULSE™ tube (Pressure Biosciences, Gaithersburg, Md.) with 1.2 mls of buffer RLT (supplied in the Qiagen RNeasy kit). PULSE™ tubes were inserted into the PCT-NEP2017 pressure cycler and the sample was extracted using the following parameters: room temperature; 5 pressure cycles of 35 Kpsi with pressure held for 20 seconds at the top and bottom of each cycle. After pressure extraction the buffer was removed and used to process the remaining tapes used to strip that site; the buffer was then processed according to the standard Qiagen RNeasy protocol for the collection of larger RNAs (>200 nucleotides) by application to a purification column to which large RNA molecules (i.e. mRNAs) bind, while the column flow-through is saved for microRNA purification. The column flow-through, which contains miRNA separated from mRNA, is processed according to the Qiagen miRNA purification procedure (on the world wide web at qiagen.com/literature/protocols/pdf/RY20.pdf) to purify the microRNA. RNA from the 2 sites stripped on each subject was pooled to create a single sample from each subject.

RNA isolation using MELT total nucleic acid protocol. Tapes were extracted in a 2 ml Eppendorf tube with 192 ml MELT buffer plus 8 ml of MELT cocktail and vortexed for 10 minutes at room temperature. The MELT lysates were transferred to the dispensed binding bead master mix after spinning down for 3 minutes at >10,000*g and washed with 300 ml of Wash Solution 1 and 2. RNAs were eluted in 100 ml of elution solution.

Quantitation of mRNA. Experimental data is reported as the number of PCR cycles required to achieve a threshold fluorescence for a specific cDNA and is described as the “Ct” value (Gibson, Heid et al. 1996; Heid, Stevens et al. 1996; AppliedBiosystems 2001). Quantitation of total RNA mass was performed as previously described (Wong, Tran et al. 2004). Briefly, RNA mass recovered from tapes is determined by using quantitative RT-PCR with reference to a standard curve (Ct, actin vs. log [RNA]; AppliedBiosystems 2001) created from commercially purchased human spleen total RNA. The average of 6 replicate Ct, actin values was used to calculate the concentration of RNA in a sample with reference to the standard curve.

RNA amplification and array hybridization. RNA was isolated by the Multi-Enzymatic Liquefaction of Tissue method (Ambion, Austin, Tex.) and amplified using the WT-Ovation pico amplification system (NuGen, San Carlos, Calif.). The amplified RNA was hybridized to Affymetrix U133 plus 2.0 microarray and data were processed and analyzed using R from Bioconductor.

Preprocessing GeneChip Data. The image files from scanning the Affymetrix GeneChips with the Affymetrix series 3000 scanner will be converted using GCOS software (Affymetrix) to “CEL” format files. Normalization of CEL files will be carried out using software from the Bioconductor suite (on the world wide web at bioconductor.org). In particular, a robust multiarray analysis with adjustments for optical noise and binding affinities of oligonucleotide probes (Wu et al., 2006; and Wu et al., 2004) as implemented by the function “just.gcrma” in the “gcrma” package will be used to normalize the GeneChip Data.

Statistical Approach for Microarray Data Analysis. Two generic statistical problems are addressed in this proposal: (i) identifying genes that are differentially expressed in different classes of lesions (i.e. melanoma versus non-melanoma lesions) and (ii) forming (and evaluating) rules for classification of melanoma and non-melanoma lesions into groups based on gene expression data.

The most important grouping divides melanoma from non-melanoma on the basis of biopsy results. The methods that will be used to address the problems identified above are now standard in the statistical evaluation of microarray data (for reviews see Smyth et al., 2003; and Lee, 2004)). These methods have been applied by others to data from Affymetrix arrays to study gene expression in prostate cancer (Stuart et al., 2004), to characterize changes in gene expression subsequent to HIV infection (Mitchell et al., 2003), and to develop a high throughput genotyping platform (Wolyn et al., 2004; and Borevitz et al., 2003). For identifying differentially expressed genes, permutation based estimates of false discovery rates (reviewed in Efron et al., 2002) are preferred. Scripts for the R quantitative programming environment were developed to implement these methods in our previous work, but will likely use or adapt the “siggenes” package from the Bioconductor suite in this project. The development of classification rules will rely on resampling methods (k-fold cross-validation, the 632 plus bootstrap, and/or bagging (Hastie et al., 2001) applied to the naive Bayes classifier and the nearest shrunken centroid classifier (Tibshirani et al., 2002) and the support vector machine (SVM) which both performed well in classifying prostate tissues as malignant or benign, used in our previous work. The implementation likely to be used is to perform k-fold cross-validation. Within each of the k train/test cycles an initial screen of the training data for differentially expressed genes is performed and genes are ordered according to their posterior probability of differential expression. Naive Bayes and nearest shrunken centroid classifiers based on the r genes with the highest posterior probability of differential expression are formed choosing enough values of r between 1 and 1024 to allow accurate interpolation of the classification error rate. The “one se rule” (Brieman et al., 1984) is applied to the error rates for the test sets to choose the classifier that minimizes the error rate. For SVM, an internal 632+ bootstrap is applied to each training sample to select the number of genes to be used in forming the classifier. The “1 se rule” error rates from the k test sets are used to characterize the classification accuracy.

QC metrics for RNA, amplified cDNA and microarray data. Following informed consent, the suspicious pigmented lesion was tape stripped using EGIR and then biopsied as per standard of care. The resulting RNA isolated from the EGIR tape was amplified and profiled on the Affymetrix U133 plus 2.0 GeneChip. Microarray data were normalized by the GCRMA algorithm. To assure high quality of microarray data are generated, QC metrics were established for RNA, amplified cDNA and microarray data. The quality of RNA was assessed by capillary electrophoresis using the Experion system (Biorad, Hercule, Calif.) and RNA with at least one visible 18S rRNA was further processed for RNA amplification. The amplified cDNA was quantified by the Nanodrop system and quality of the amplified cDNA was also assessed by the Experion system. The yield of the amplified cDNAs greater than 5 mg and the average size distribution of the cDNAs greater than 750 nt were carried forward for microarray hybridization. Quality of the array data was further assessed using simpleaffy program in R and the array data with scaling factor less than 5.0 and % present call greater than 30% were used for further data analysis.

Class Modeling-PAM. After passing the array data QC, 14 melanomas, 40 dysplastic nevi and 12 normal skin specimens were further analyzed. First, gene expression values less than 50 across all samples were filtered out and 16716 probesets were tested. These 16716 probesets were subjected to a statistical analysis for differentially expressed genes among melanomas, dysplastic nevi and normal skin using ANOVA (p<0.05), multiple testing correction algorithm (Westafall and Young permutation) and false discover rate (FDR) of 0.05. As indicated above, of the original 117 genes, an 89 gene panel (Table 2) was found to be a potential melanoma classifier. Further testing identified a 5-gene classifier (Table 3), a 30-gene classifier (Table 4) that includes newly identified genes, a 20-gene classifier (Table 5) that includes newly identified genes, and a 19-gene classifier that includes newly identified genes, which may all be used to discriminate melanomas from atypical nevi. The genes and respective classifier panels were analyzed using the Prediction Analysis of Microarrays (PAM) software freely available from Stanford University (Stanford, Calif.).

The PAM software uses a modification of the nearest centroid method, which computes a standardized centroid for each class in a training set. This refers to the average gene expression for each gene in each class divided by the within-class standard deviation for that gene. Nearest centroid classification takes the gene expression profile of a new sample, and compares it to each of these class centroids. The class, whose centroid it is closest to, in squared distance, is the predicted class for that new sample.

These genes were all subjected to a hierarchical clustering analysis and the melanoma specimens grouped together and were clearly distinguished from dysplastic nevi and normal skin. In addition, there are three distinct classes of dysplastic nevi; one is grouped together with normal skin and the second one was in between normal skin and melanomas, while the third one was grouped together with melanomas. These data suggest stratum corneum RNA, harvested by tape stripping with EGIR (epidermal genetic information retrieval), can be used to distinguish melanoma from dysplastic nevi in suspiciously pigmented lesions.

The analysis of the genes as potential melanoma classifiers to discriminate between melanomas and dysplastic nevi was performed using t-test (p<0.01), FDR (0.05) and 2-fold difference between melanomas and dysplastic nevi.

### Example 3

### Assessment of Performance of Multi-Gene Biomarker for Melanoma Detection

### Custom TaqMan OpenArray (OA) qPCR Plate (See Figure to the Right)

The OpenArray real-time PCR system (Life Technologies, Carlsbad, Calif.) was chosen for investigation. Each OA contained 48 sub-arrays (4×12). Each sub-array contained 18 target genes in triplicate, including genes procured from discovery studies to generate a melanoma classifier, and 3 internal genes (Wachsman et al., Brit. J. Dermatol 164:797 [2011]).

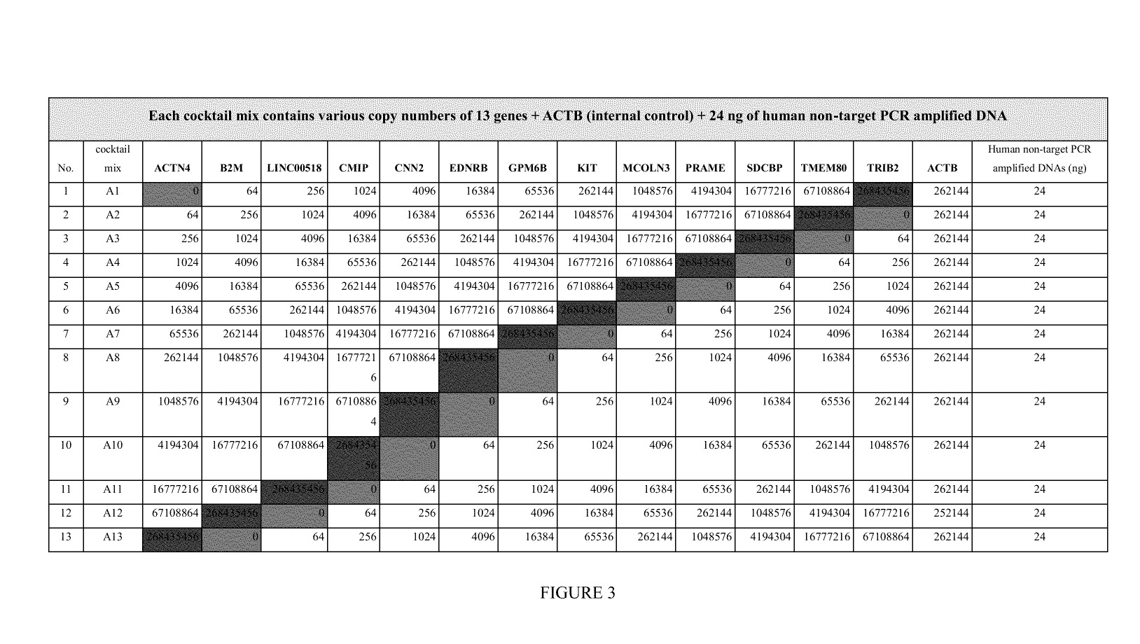

Latin Square Experimental Design and Method (See FIG. 3)

13 cloned human genes were used, including: ACTN4, B2M, LINC00518, CMIP, CNN2, EDNRB, GPM6B, KIT, MCOLN3, PRAME, SDCBP, TMEM80 and TRIB2.

Each of the 13 genes were pooled into groups and diluted to copies of 0, 64, 256, 1024, 4096, 1.6E+04, 6.6E+04, 2.6E+05, 1.0E+06, 4.2E+06, 1.7E+07, 6.7E+07 and 2.7E+08.

In every qPCR experiment, 13 groups of genes in 13 different copies were assayed in triplicate in the presence of a complex human background, produced from EGIR-harvested normal human skin that was amplified with 20 primer sets for non-target mRNAs, and the ACTB internal control gene. After assaying on the OA platform, the qPCR data were de-convoluted to ascertain sensitivity and linear dynamic range for each of the 13 genes.

Gene Expression Profiling of EGIR Specimens

Specimens were collected at 8 clinical sites in US and Spain and shipped and stored at −80° C. Total RNA was extracted using MELT (Ambion, Inc.). Only part of tape demarcated over pigmented lesion were used for analysis, and RNA product was pooled from all 4 tapes (yield 500-2,000 pg).

For microarray analysis, RNA was amplified using the WT-Ovation FFPE kit (NuGen, Inc.). The microarray assay used was the U133 plus 2.0 GeneChip (Affymetrix, Inc.) Data was quality checked with Simpleaffy and normalized with the GCRMA algorithm (Bioconductor).

For qRT-PCR assays, RNA was pre-amplified with 14 cycles of PCR using a pool of 18 TaqMan primer/probe-sets (ABI, Inc). The qPCR data was normalized with 3 internal controls using geometric mean.

Class Prediction Modeling

TreeNet® (Salford Systems, Inc.) was used to generate a prediction model from the training dataset established for each platform (i.e. OA and microarray) for the gene targets selected from the discovery research. An independent test dataset was used to validate the class prediction model (i.e., classifier) established from the training dataset.

Clinical Protocol

Inclusion criteria: Subjects were 18 or older and had pigmented skin lesion that were suspicious for melanoma and required biopsy. Lesion size was 4 mm or greater, and if multiple lesions were present, there must have >4 mm of separation between them.

Exclusion criteria: Exclusion criteria include; the presence of a lesion that is ulcerated, bleeding or weeping; use of topical moisturizer or sunscreen on lesion sites within 24 h; and/or allergy to tape or latex.

Procedures

Lesions were tape stripped using all 4 tapes in kit. The lesion edge was demarcated on the tape, if the lesion was smaller than 17 mm in diameter. Lesions were biopsied as per standard of care. Biopsy specimens were reviewed by primary and central dermatopathology.

Purpose 1: Ascertain Adequacy of EGIR-Harvested Normal Skin RNA, Pre-Amplified with the Non-Target Primer Pool, as a Complex Background for OA qPCR Assays

Strategy

EGIR-harvested RNA from human normal skin was pre-amplified by 14 cycles of PCR in separate reactions with the TaqMan “target” and “non-target” primer pools. qPCR analyses were performed on the 7900HT system (ABI, Inc.) on each of these 2 reaction pools for expression of each of the 13 target genes.

Results

An average of 13.05 Ct (˜8500-fold) difference was observed for the 13 target genes when the two pre-amplified normal skin pools are compared (see Table 1 below), excepting LINC00518 and PRAME. LINC00518 and PRAME are expressed in melanoma, but not in normal skin.

TABLE 1

| | | EGIR tape-stripped Normal Skin RNA | | | | |
|---|---|---|---|---|---|---|
| | | PCR | | PCR | | |
| | | Pre-Amp | | Pre-Amp | | |
| | | with 18 | | with 20 | | |
| | | TaqMan | | TaqMan | | |
| | | target | | non-target | | |
| | | gene primer | | gene primer | | Ct difference of |
| | | pool | | pool | | Pre-Amp with 18 |
| | | Mean | | Mean | | targets and 20 non- |
| No. | Gene | Ct | stdev | Ct | stdev | targets |
| 1 | Actin4 | 19.74 | 0.07 | 32.82 | 0.14 | 13.07 |
| 2 | B2M | 17.90 | 0.02 | 31.08 | 0.00 | 13.18 |
| 3 | CMIP | 24.00 | 0.03 | 39.41 | 0.70 | 15.42 |
| 4 | CNN2 | 20.96 | 0.04 | 34.44 | 0.61 | 13.48 |
| 5 | EDNRB | 25.33 | 0.06 | 39.10 | | 13.77 |
| 6 | GPM6B | 24.71 | 0.01 | 33.96 | 0.58 | 14.24 |
| 7 | Kit | 23.89 | 0.06 | 37.87 | 0.53 | 13.97 |
| 8 | MCOLN3 | 26.25 | 0.06 | 38.50 | | 12.25 |
| 9 | SDCBP | 25.26 | 0.07 | 39.12 | 1.19 | 13.86 |
| 10 | TMEM80 | 27.24 | 0.02 | 37.49 | 0.71 | 10.25 |
| 11 | TRIB2 | 23.81 | 0.03 | 37.84 | 0.86 | 14.03 |
| 12 | LINC00518 | 29.47 | 0.17 | 38.12 | | 8.65 |
| 13 | PRAME | 34.90 | 0.61 | 38.23 | 0.86 | 3.33 |

These results demonstrate that the 20 non-target pre-amplified genes in normal skin RNA can serve as a complex background for the Latin Square experiment.

Purpose 2: Characterize Sensitivity, Linear Dynamic Range and Reproducibility of the OA Platform.

Strategy

The above described Latin Square experiment was performed, targeting 13 genes in a complex background.

Results

Beta-actin (ACTB) served as an internal control gene (spike-in of 2.6E+05 copies). The mean Ct was 18.82±0.29 (ranged: 18.53˜18.98) (see Table 2). CV was 1.54%.

TABLE 2
qPCR assay of spike-in ACTB gene shows negligible fluctuation in a complex
background

| | cocktail mix | | | | | | | | | | | | |
|---|---|---|---|---|---|---|---|---|---|---|---|---|---|
| | LS-1 | LS-2 | LS-3 | LS-4 | LS-5 | LS-6 | LS-7 | LS-8 | LS-9 | LS-10 | LS-11 | LS-12 | LS-13 |
| ACTB | 18.53 | 18.62 | 18.70 | 18.75 | 18.75 | 18.76 | 18.76 | 18.78 | 18.83 | 18.86 | 18.93 | 18.96 | 18.98 |
| mean Ct | | | | | | | | | | | | | |

These data indicate that the internal control ACTB gene does not fluctuate among the 13 Latin Square “cocktail mixes” in the complex background.

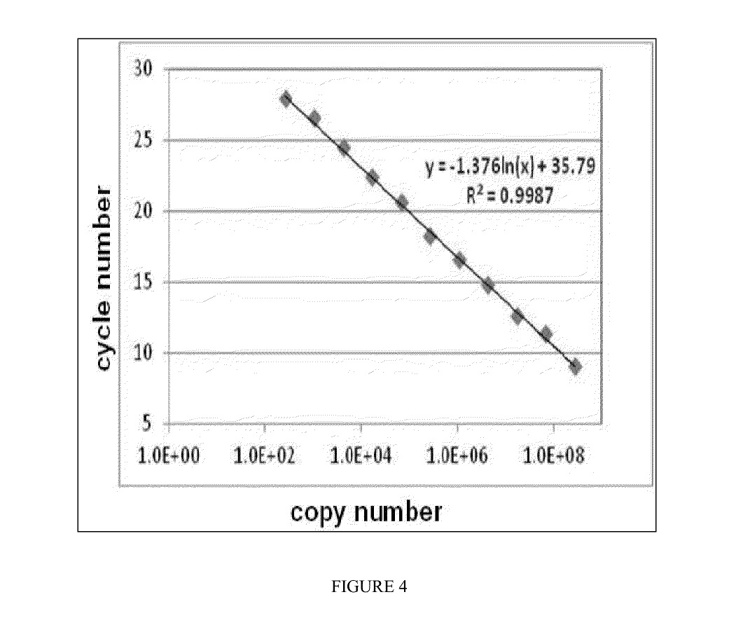

The limit of detection was found to range between 256 and 1,024 copies Data for the CMIP gene (shown in FIG. 4) show that it can be detected above background with as little as 256 copies

In addition, OA-based qPCR exhibited a 5-log of linear dynamic range (shown at Table 3).

TABLE 3
Amplification efficiency of the 13 genes on the OpenArray plate

| | | | | Amplification |
|---|---|---|---|---|
| No. | Gene | slope | R\hat{ }(2) | efficiency |
| 1 | ACTN4 | 2.982 | 0.989 | 116.45 |
| 2 | B2M | 2.768 | 0.989 | 129.78 |
| 3 | LINC00518 | 3.304 | 0.992 | 100.74 |
| 4 | CMIP | 3.194 | 0.999 | 105.64 |
| 5 | CNN2 | 3.012 | 0.996 | 114.80 |
| 6 | EDNRB | 3.231 | 0.993 | 103.96 |
| 7 | GPM6B | 3.279 | 0.992 | 101.83 |
| 8 | KIT | 3.009 | 0.995 | 114.92 |
| 9 | MCOLN3 | 3.304 | 0.989 | 100.74 |
| 10 | PRAME | 3.104 | 0.987 | 109.98 |
| 11 | SDCBP | 3.403 | 0.992 | 96.72 |
| 12 | TMEM80 | 3.104 | 0.993 | 109.98 |
| 13 | TRIB2 | 3.272 | 0.989 | 102.13 |

Reproducibility was CV<3.0, based on 13 samples analyzed in triplicate for each of 13 gene targets.

The mean amplification efficiency was 108.3±9.0 (range: 96.7˜129.8). These data suggest the amplification efficiency of qPCR assays on the OA plate is not affected in the presence of background DNA.

Purpose 3: Comparatively Assess the Performance of a Multi-Gene Biomarker for Melanoma on the TaqMan OA and Microarray Platforms

Strategy: Develop Class Prediction Models for the Microarray and OA Platforms

TreeNet algorithm was applied to training dataset expression data, generated with each platform, for each of the 13 target genes. 35 melanomas and 35 nevi were used as a training dataset. The test dataset consisted of 22 melanomas and 26 nevi.

Results

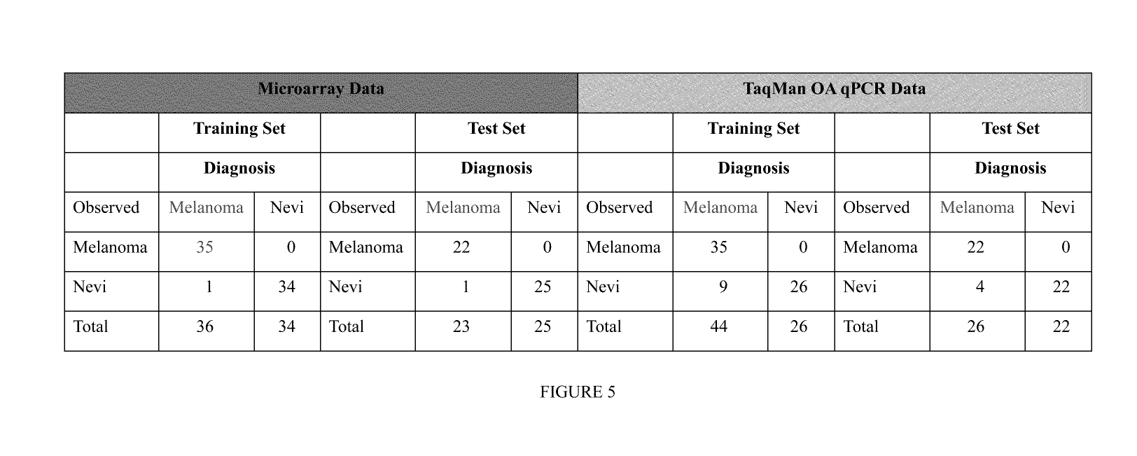

Training: For both the microarray and OA, the 13-gene classifier classified melanoma as distinct from nevi with 100% sensitivity (see FIG. 5).

Testing:

Using the microarray platform, sensitivity was found to be 100% and specificity was found to be 96%. In ROC analysis, AUC>0.921

Using the qRT-PCR platform: 100% sensitivity was found to be 100% and specificity was found to be 85%. In ROC analysis, AUC>0.912

### Example 4

### Identification of Melanoma Gene-Classifier Pairs

The study protocols were reviewed and approved by the various Institutional Review Boards that submitted specimens for the study. Study subjects provided written consent prior to participation. The study was conducted according to the declaration of the Helsinki principles.

Inclusion Criteria:

Subjects were eligible and were recruited into the study if they were at least 18 years of age and had a pigmented lesion of at least 4 mms in diameter that was clinically suspicious for melanoma. All patients underwent a standard of care biopsy with histologic evaluation. If the patient had multiple clinically suspicious lesions, they had to be at least 4 mms apart to be included in the study.

Exclusion Criteria:

Subjects could not participate in the study if they had used topical medications (corticosteroids, alpha-hydroxyacids, retinoids, antibiotics) or systemic steroids within 30 days of beginning the research study or if the subject had a generalized skin disorder not related to skin cancer such as psoriasis, a photosensitivity dermatitis or eczema; allergy to tape or latex rubber. Subjects were also excluded if they were currently participating, or had participated in the 30 days prior to the study, in an investigational (OTC, RX or device) study; or had clinical findings that the Investigator felt were suggestive of an advanced stage lesion (e.g. ulcerated, bleeding, oozing).

Lesion Selection Criteria:

Lesions suspicious for melanoma and selected for biopsy were tape striped prior to biopsy. Advanced stage lesions (e.g. ulcerated, bleeding, oozing) that may be physically damaged by the tape stripping procedure were not included.

Tape-Stripping Procedure:

Tape stripping was performed before all biopsy procedures. The tape was applied to the site and briskly rubbed with the blunt rounded end of a marker or plastic test tube in a circular motion. A minimum of 15 circular motions were completed before the tape was removed. The border of the lesion was demarcated on the tape with a surgical marker. A total of 4 tapes were used to sample all sites. After sampling, the tapes were stored at −20° C. or below within 10 minutes of stripping. The tapes were shipped on dry ice, by express mail, to be analyzed within one week of tape stripping.

Biopsy:

After the tape-stripping procedure was completed, the lesion was biopsied or excised according to standard clinical practice. The biopsy was a standard of care procedure that would have been conducted regardless of the research. All tissues removed were fixed in formalin and sent to a histopathology laboratory, where they were embedded in paraffin and sectioned for histopathological analysis. Selected tissue slides (either the actual slides used for diagnosis or diagnostically equivalent slides) were sent to a dermatopathologist contracted by DTI for central review. Three separate dermatopathologists (the site pathologist and DTI pathologists) were involved in the reading of each case. Further inclusion criteria for the study required histologic agreement between all three dermatopathologists involved in the study.

Quantitation of Gene Expression:

The area of the pigmented lesion was delineated on each piece of tape by the collecting site, and shipped on dry ice to a laboratory via overnight courier. The outlined lesional portion of each tape was manually dissected and the four tapes for each lesion were pooled for further processing. Total RNA was isolated for each sampled pigmented lesion and used for cDNA synthesis; that cDNA was subsequently used for pre-amplification and quantitative real-time PCR (qrt-PCR) using standard Taqman chemistry. RNA was isolated using Ambion MELT™ (Life Technologies, Foster City Calif.); cDNA synthesis was performed using SuperScript VILO™ (Life Technologies); pre-amplification was performed using a custom pool of primers and probes for all genes assayed (Life Technologies). Quantitation of gene expression levels was performed using the QuantStudio OpenArray™ system (Life Technologies). Expression levels of two candidate signature genes for each specimen were normalized to the geometric mean of expression levels of three housekeeping genes (ACTB, B2M, and PPIA) for that specimen.

Statistical Analysis:

An optimal classification signature was created separately for each of 2 independently ascertained datasets. The selection of genes for inclusion in the classification signature was performed using stochastic gradient boosting analysis coupled with bootstrap logistic decision tree modeling (TreeNet™, Salford Systems, San Diego Calif.) and logistic regression (R: A Language and Environment for Statistical Computing. R Development Core Team, R Foundation for Statistical Computing. Vienna, Austria. 2011. http://www.R-project.org) on the gene classifier list described in Example 3. Bootstrapping is a machine learning technique that iteratively divides the dataset into random training and test classes and assesses classification performance for each iteration. TreeNet was used to create the classification algorithm and to determine each gene's relative importance within the signature. The classification algorithm computes a score between 0 and 1 for each specimen based on the measured gene expression inputs. Sensitivity, specificity, NPV, PPV, Receiver Operator Characteristic (ROC) curves, and AUC (area under the ROC curve) were calculated using R.

Results

Gene Expression Classification of Melanoma: A high accuracy gene classification signature was used as a starting point for validation (see Example 3). In order to further understand the signature, the relative importance of each gene within the total profile was assessed.

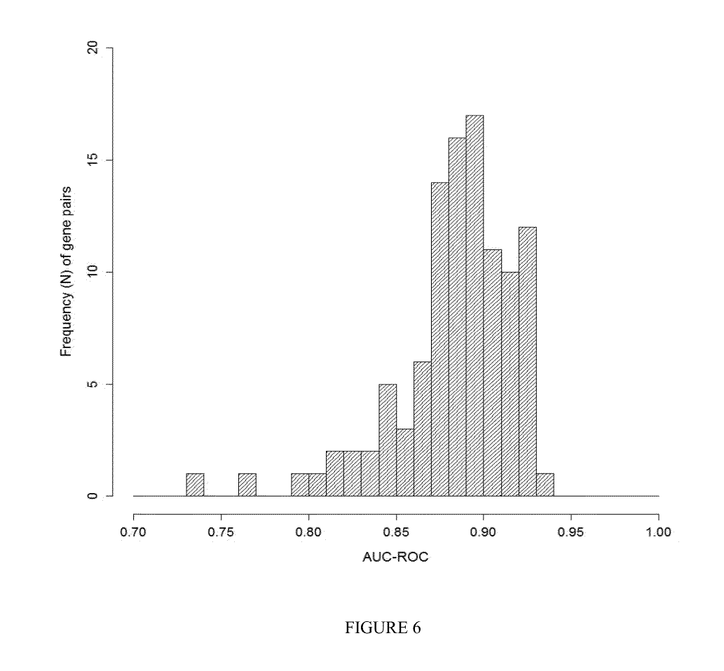

All 105 pairwise combinations from within the previous 15 gene signature were investigated for accuracy by bootstrap multivariate logistic regression using a 140-specimen set, including 69 melanomas, 51 nevi and 20 other non-melanocytic neoplasms of the skin (keratoses, solar lentigos, and basal cell carcinoma). As shown in FIG. 6, the mean AUC of each gene pair's iterations ranged from a low of 0.72 to a high of 0.94 (an AUC of 1.00 is a perfect classification system). The gene pairs with average AUCs above 0.90 were further investigated using TreeNet analysis (FIG. 6).

The top-performing gene pair in the logistic regression studies (CMIP and LINC00518) was also the best performing pair using the TreeNet classification algorithm. The signature using two genes was then validated in a newly tested set of 64 cases consisting of 44 melanomas and 22 nevi.

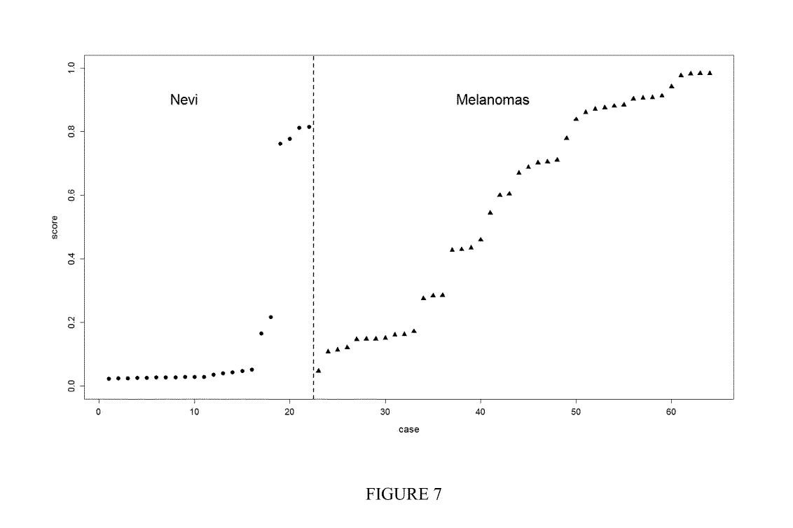

Table 4 gives the 2×2 table of disease status versus expression signature classification; FIG. 7 shows a graphic distribution of the cases' scores.

TABLE 4
Pathology Diagnosis vs. Expression Signature
Classification, Classifier A on Set B

| | | Pathology Diagnosis | |
|---|---|---|---|
| | | Melanoma | Nevi |
| Gene Expression | Melanoma | 43 | 16 |
| Classification | Non-melanoma | 1 | 6 |

| | |
|---|---|
| Sensitivity | Specificity |
| 43/44 = 97.7% | 16/22 = 72.7% |

The resulting sensitivity was 97.6% and specificity was 72.7%. The NPV was 94% at the study prevalence of 66% melanoma, and was 99.6% at an adjusted melanoma prevalence of 10% to more closely reflect what is reported for general dermatology practices.

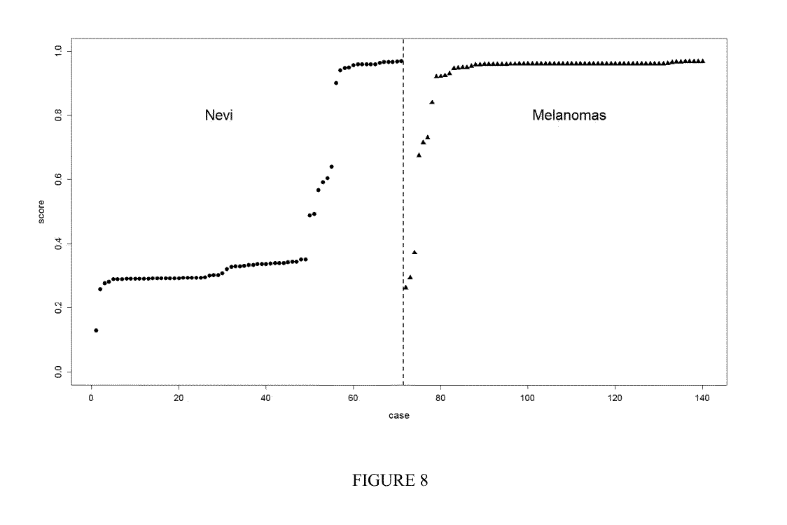

A “converse validation” was subsequently performed by similarly using the same two-genes to create a classification algorithm from the set of 64 cases (Set B) and applying it to the initial independent set of 140 cases (Set A). Comparable validation accuracies were seen with sensitivity of 97% and specificity of 68%. Table 5 gives the 2×2 table of disease status versus expression signature classification; FIG. 8 shows a graphic distribution of the cases' scores.

TABLE 5
Pathology Diagnosis vs. Expression Signature
Classification, Classifier B on Set A

| | | Pathology Diagnosis | |
|---|---|---|---|
| | | Melanoma | Non-melanoma |
| Gene Expression | Melanoma | 67 | 48 |
| Classification | Non-melanoma | 2 | 23 |

| | |
|---|---|
| Sensitivity | Specificity |
| 67/69 = 97.1% | 48/71 = 67.6% |

The NPV was 88% at the study prevalence of 73% melanoma, and was 99.4% at an adjusted melanoma prevalence of 10% to more closely reflect what is reported for general dermatology practices (FIG. 8).

In this study some cases were eliminated because insufficient mRNA for one of the two signature genes was isolated. Interestingly while it is recognized that distinct subtypes of melanoma have distinct genetic aberrations, the two gene signature of CMIP and LINC00518 was successful in identifying melanomas from both sites of chronic sun damage as well as melanomas from areas of intermittent sun exposure. One case diagnosed as melanoma by histopathology but not by the two gene classifier of CMIP and LINC00518 was a lentigo maligna melanoma in situ.

The two genes CMIP and LINC00518, with a combined ability to discriminate between melanoma and non-melanoma cases with relatively high accuracy, represent a novel biomarker discovery arrived at through top-down genomic data mining. Importantly the ability to reduce the signature to 2 genes will allow the assay to be performed using automated quantitative PCR assay with rapid turn-around time.

In vivo, the mRNA from the critical signature genes produced by melanocytes is phagocytized by epithelial cells. The tape stripping method is non-invasive only removing a superficial layer of epithelial cells and does not result in any wound or require any down time from physical activity or work for the patient. While the overall morbidity associated with skin biopsy is quite low, each biopsy may result in decreased physical activity including time off from work, anxiety associated with having a procedure done and considerable cost. In the case of dysplastic nevus syndrome patients this procedure would allow for the simultaneous evaluation of a greater number of atypical nevi where as doing more than 3 or 4 excisional biopsies at one time could result in considerable discomfort for the patient and require a significant amount of wound care.

Assuming an NNT (“number needed to treat”, used to measure physician accuracy in assessing pigmented skin lesions) between 10 and 30 for pigmented lesions, a single patient with 2 melanomas in their lifetime may have 20 to 60 biopsies to identify the 2 malignant neoplasms. Furthermore, some pigmented lesions on the face, genitalia or other sensitive anatomic locations may be difficult to biopsy and may result in unsightly scars. The high sensitivity (97.6%) of the assay accompanied with a specificity of 72.7%, could significantly decrease the number of unnecessary biopsies, therefore decreasing the NNT to a value of 2. This would result in a significant improvement in quality of life particularly for dysplastic nevus syndrome patients who may have hundreds of clinically atypical nevi and more than one melanoma in their lifetime. This could also result in a significant savings in health care expenditures.

Clinical, epidemiologic and molecular evidence suggests some histologically malignant melanocytic lesions exhibit clinical behavior consistent with benignity. An analysis as described herein providing molecular genetic data on the lesion maybe an additional useful data point and allow for further refinement of the final diagnosis. Six of twenty-two cases with a histologic diagnosis of nevus had and were found to have a score based on the two gene classifier of CMIP and LINC00518 consistent with a diagnosis of melanoma. Interestingly among the six cases, three had a histologic reading of dysplastic nevus with severe atypia. Unfortunately, it cannot be determined whether these three cases were truly false positives or actually early melanomas. Regardless, the specificity of 72.7% in the validation set is significantly higher than the specificity of other currently used methods for evaluation of pigmented melanocytic neoplasms including dermoscopy (reference on specificity of dermoscopy).

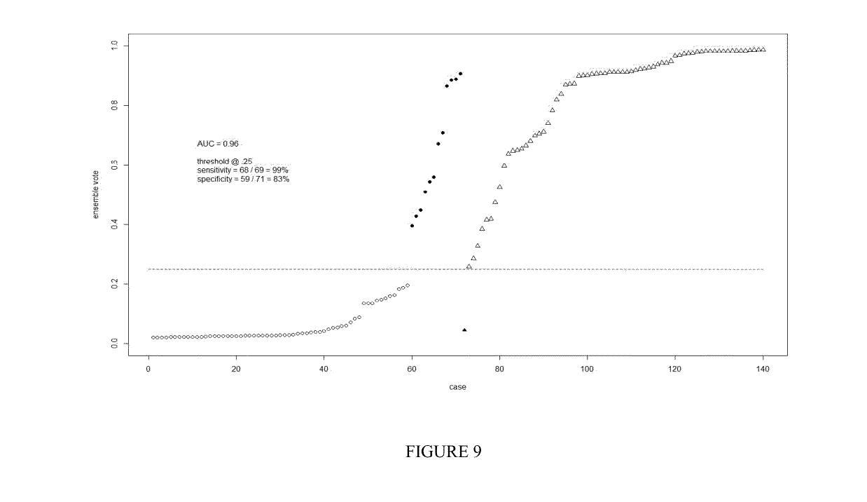

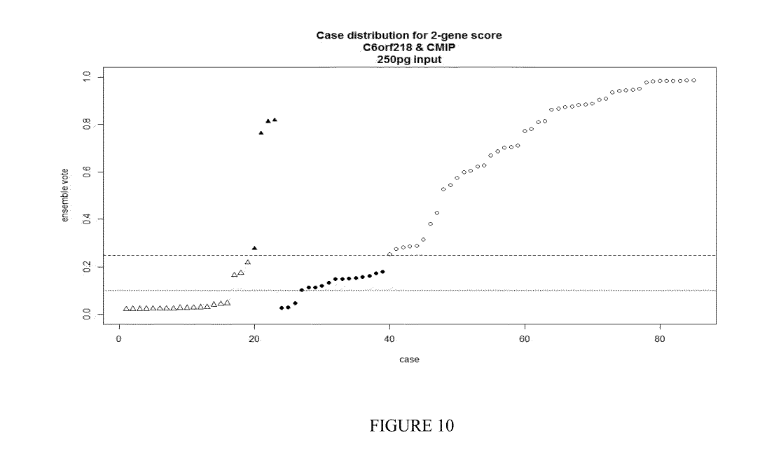

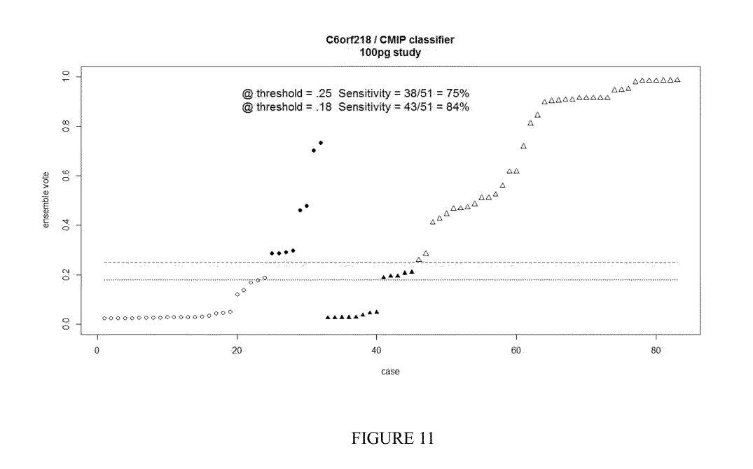

The sensitivity of the assay at for varying amounts of input RNA is shown in FIGS. 9, 10 and 11. 1000 pg, 250 pg and 100 pg of RNA was tested. For 1000 pg input RNA, the AUC was found to be 0.96, the sensitivity was found to be 99% and the specificity was found to be 83% (FIG. 9). For 250 pg input RNA, the sensitivity was found to be 95%, while the specificity was found to be 70%. For 100 pg input RNA, at a threshold of 0.25, the sensitivity was 75%, whereas at a threshold of 0.18, the sensitivity was found to be 84%.

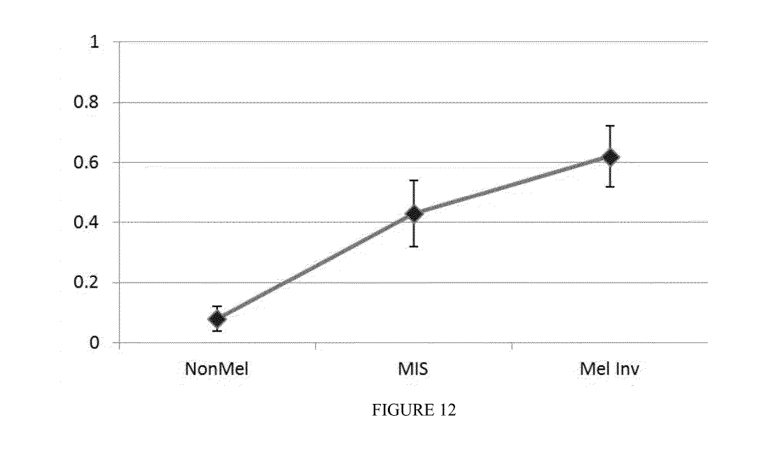

The mean of the algorithmic score is shown for each set of specimens in the validation study for the gene pair combination of LINC00518 and CMIP in classifying non-melanoma, melanoma in situ, and invasive melanoma, with error bars indicating the 95% confidence interval of that mean is shown FIG. 12. A one-way analysis of variance shows a significant difference of the algorithmic score among the 3 groups, with a p-value<<0.0001. While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for diagnosing melanoma in a subject, comprising:
(a) detecting nucleic acids expressed by two or more genes comprising: ACTN4, LINC00518, CMIP, TTC3, TMEM80, HLA-C, or PRAME in a sample of a skin lesion from the subject; and
(b) characterizing the skin lesion as melanoma, based on the detection of the nucleic acids, thereby diagnosing the presence or absence of melanoma.

2. The method of claim 1, provided that the detecting comprises determining the level of expression of the nucleic acids.

3. The method of claim 1, provided that at least one of the two or more genes is LINC00518.

4. The method of claim 1, provided that the two or more genes consist of LINC00518 and CMIP.

5. The method of claim 4, provided that at least one nucleic acid comprises RNA.

6. The method of claim 1, further comprising amplifying nucleic acid obtained from the sample prior to detecting.

7. The method of claim 1, provided that the sample is obtained by applying an adhesive tape to a target area of skin in a manner sufficient to isolate the sample adhering to the adhesive tape.

8. The method of claim 7, provided that about one to ten adhesive tapes or one to ten applications of a tape are applied and removed from the skin.

9. The method of claim 7, provided that the tape comprises a rubber adhesive on a polyurethane film.

10. The method of claim 1, further comprising administering a treatment regimen based on the diagnosis.

11. The method of claim 1, provided that nucleic acid, or an amplification product thereof, is applied to a microarray.

12. The method of claim 1, provided that an expression profile is detected using a microarray.

13. The method of claim 1, provided that the sample is obtained from a biopsy sample taken at the site of the skin lesion or surrounding margin.

14. A method of distinguishing melanoma from dysplastic nevi or normal pigmented skin in a subject, comprising:
(a) detecting nucleic acids expressed by two or more genes comprising: ACTN4, LINC00518, CMIP, TTC3, TMEM80, HLA-C, or PRAME in a sample from the subject; and
(b) characterizing the skin sample as melanoma, dysplastic nevi or normal pigmented skin, based on the detection of the nucleic acids, thereby distinguishing melanoma from dysplastic nevi or normal pigmented skin in a subject.

15. The method of claim 14, provided that detecting comprises determining the level of expression of the nucleic acids.

16. The method of claim 14, provided that at least one of the two or more genes is LINC00518.

17. The method of claim 14, provided that the two or more genes comprise LINC00518 and CMIP.

18. The method of claim 14, provided that the sample is obtained by applying an adhesive tape to a target area of skin in a manner sufficient to isolate the sample adhering to the adhesive tape.

19. A system for diagnosing melanoma in a subject, comprising:
(a) a computer processing device, optionally connected to a computer network; and
(b) a software module executed by the computer processing device to analyze nucleic acids from two or more genes comprising: ACTN4, LINC00518, CMIP, TTC3, TMEM80, HLA-C, or PRAME in a sample from a skin lesion, to a standard or control.

20. The system of claim 19, wherein LINC00518 and CMIP are analyzed by the system.
